(19) United States
(12) Patent Application Publication
(10) Pub. No.: US 2004/0097401 A1
(43) Pub. Date: May 20, 2004

(54) Lysine in therapeutic angiogenesis, particularly in treating ischaemic conditions

(76) Inventor: Debatosh Datta, Kolkata (IN)

Correspondence Address: NOTARO AND MICHALOS, 100 DUTCH HILL ROAD, SUITE 110, ORANGEBURG, NY, 10962-2100, US

(21) Appl. No.: 10/294,308

(22) Filed: Nov. 14, 2002

(51) Int. Cl. A61K 38/00; A61K 31/198

(52) U.S. Cl. 514/2; 514/564; 514/565

(57) ABSTRACT

Present invention features methods for induction of angiogenesis by administration of lysine (l-&d-) or lysine oligomers (molecular weight approx between 500 and 2500), both homo and hetero-oligomers, consisting of either l-or d- or both enantiomers.

Induction of Angiogenesis by the methods of the invention can be use in therapeutic angiogenesis, in, for example, treatment of ischaemic conditions and syndromes, such as chronic wounds (e.g diabetic wounds and ulcers, bed sores and other pressure sores, burns of various degrees and extents etc.) as well as coronary and cerebral ischaemia and peripheral vascular ischaemic conditions. Induction of angiogenesis by the described methods also will be useful in inducing/enhancing radiosesitivity in some solid tumors.

### DESCRIPTION

1. Field of Invention

The invention relates generally to the field of regulation of angiogenesis and vasculogenesis, particularly growth of new vasculatre.

2. Background of the Invention

Angiogenesis is a process involved in the growth of blood vessels. Angiogenesis is the process by which new vessels are formed from extant capillaries and the factors that regulate this process are important in embryonic development and contribute to pathologic conditions such as tumor growth, diabetic retinopathy, rheumatoid arthritis etc. (U.S. Pat. No. 5,318,957; Yancopoulos et al. (1998) Cell 93:661-4; Folkman et al. (1996) Cell 87:1153-5).

Angiogenesis involves the proliferation of endothelial cells. Endothelial cells line the walls of the vessels, while capillaries are comprised almost entirely of endothelial cells. The angiogenic process involves not only increased endothelial cell proliferation but also comprises a cascade of additional events including protease secretion by endothelial cells degradation of the basement membrane, migration through surrounding matrix, proliferation, alignment, differentiation into tube-like Structures, and synthesis of a new basement membrane (Folkman et al. (1996) Cell 87:1153-5).

Several angiogenic agents with different properties and mechanisms of action are well known in the art, e.g. acidic and basic fibroblast growth factor (FGF), transforming growth factor alpha (TGF-alpha) and beta (TGF-beta), Tumour Necrosis Factor (TNF), Platelet derived growth factor (PDGF), vascular endothelial cell growth factor (VEGF), and angiogenin are potent and well characterized angiogenesis promoting agents. However, the therapeutic applicability of some of these compounds, especially as systemic agents is limited by their potent pleiotropic effects on various cell types.

Angiogenesis has been the focus of intense interest since this process can be exploited to therapeutic advantage. Stimulation of angiogenesis can aid in the healing of wounds, the vascularization of skin grafts, and the enhancement of collateral circulation, where there has been vascular occlusion or stenosis (e.g. to develop a bio-bypass around an obstruction due to coronary, carotid or peripheral arterial occlusion disease). There is an intense interest in the factors that are well tolerated by the subject, and at the same time effective in stimulation of angiogenesis in vascular-compromised tissues.

3. Related Art

Cooke J et al. (2002) Nicotine in therapeutic angiogenesis and vasculogenesis. U.S. Pat. No. 6,417,205 described the angiogenic and vasculogenic property of Nicotine and other nicotine receptor agonists.

Villablanca ((1998) Nicotine stimulates DNA synthesis and proliferation in vascular endothelial cells in-vitro; J. Appl. Physiol. 84:2089-98) studied the effects of Nicotine on endothelial DNA synthesis, DNA repair, proliferation and cytotoxicity using cultures of bovine pulmonary artery endothelial cells in vitro.

The reference Carty et al. ((1996) Nicotine and cotinine stimulate secretion of basic fibroblast growth factor and affect expression of matrix metalloproteinases in cultured human smooth muscle cells; J. Vasc. Surg. 24:927-35) demonstrate that nicotine stimulates vascular smooth muscle cells to produce fibroblast growth factor, and also upregulates the expression of several matrix metalloproteinases. The investigators propose that this data demonstrate mechanisms by which smoking may cause atherosclerosis and aneurysms.

Lipid molecules e.g. spingosine-1-phosphate (spp) has been implicated in angiogenesis and blood vessel maturation (English D, et al. (2002) Biochim Biophys Acta, 1582(1-3):228-39). SPP has been reported to be capable of inducing almost every aspect of angiogenesis.

Katada, J et al. ((2002), J Pharmacol Exp Ther 302(3):949-56) described the role of chymase, a serine protease, capable of angiotensin conversion (I to II), in the process of angiogenesis through VEGF upregulation mediated by Ang II.

Sivestre J S and Levy B I ((2002) Arch Mal Coeur Vaiss, 95(3): 189-96) described the uses of recombinant angiogenic growth factors e.g. VEGF, FGF etc. as well as drugs with proangiogenic activity in induction of functional revascularization through angiogenesis.

Kamihata H et al. ((2001) Circulation 104(9): 1046-52) described the role of bone marrow mononuclear cells' implantation in induction of angiogenesis in ischaemic rat heart model.

Yamamoto S et al. ((2001) Jpn Circ J, 65(6): 565-71) described the potential role of ultrasound therapy in inducing transmyocardial channels resulting in improved perfusion.

Kawasuji M et al. ((2000) Ann Thorac Surg., 69(4)1155-61) described, induction of therapeutic angiogenesis with intramyocardial administration of basic fibroblast growth factor.

Roles of various angiogenic growth factors all protein in nature, has been described by various workers e.g. Harrigan M R et al. (2002) Neurosurgery 50(3):589-98; Edelberg J M et al. (2002) Circulation 105(5):608-13; Freedman S B & Isner J M (2002) Ann Intern Med 136(1):54-71 etc.

### SUMMARY OF THE INVENTION

The present invention features methods for induction of angiogenesis by administration of Lysine or Lysine oligomers (up to Mol. wt. 1000) or Lysine analogue (d-isomer). Induction of angiogenesis by the methods of the invention can be used in therapeutic angiogenesis in, for example, treatment of ischaemic syndromes e.g. coronary, cerebral or peripheral arterial diseases.

One object of the present invention is to provide a method of controlling, particularly enhancing angiogenesis with NO adverse effects or with very limited adverse effects.

Another object of the present invention is to provide a method of treating diseases and ailments involving angiogenesis e.g. myocardial and cerebral infarctions, mesenteric or limb ischaemia, wounds and vascular occlusion or stenosis.

Another object of the present invention is to provide a method of enhancing angiogenesis to accelerate wound healing or the vascularization of a skin graft musculocutaneous flap or other surgically transplanted tissues or to enhance the healing of a surgically created anastomosis.

These and other objects, advantages and features of the invention will become apparent to those persons skilled in the art upon reading the details of methods of the invention and compositions used therein as more fully described below.

Other References

English, D et al. (2002) Lipid mediators of angiogenesis and the signaling pathways they initiate. Biochim Biophys Acta 1582(1-3):228-39.

Katada, J et al. (2002) Significance of vascular endothelial cell growth factor up-regulation mediated via a Chymasw-Angiotensin dependent pathway during angiogenesis in hamster Sponge Granulomas. J Pharmacol Exp Ther 302(3):949-56.

Sivestre, J S and Levy, B I (2002) Angiogenesis therapy in ischaemic disease. Arch Mal Coeur Vaiss 95(3):189-96.

Kamihata, H et al. (2001) Implantation of bone marrow mononuclear cells into ischaemic myocardium enhances collateral perfusion and regional function via side supply of angioblasts, angiogenic ligands and cytokines. Circulation 104(9):1046-52.

Yamamoto, S et al. (2001) Potential use of ultrasound in creating transmyocardial channels. Jpn Circ J 65(6):565-71.

Kawasuji M et al. (2000)Therapeutic angiogenesis with intramyocardial administration of basic fibroblast growth factor. Ann Thorac Surg 69(4):1155-61.

Harrigan M R et al. (2002) Intraventricular infusion of vascular endothelial growth factor promotes cerebral angiogenesis with minimal brain edema. Neurosurgery 50(3):589-98.

Cuevas P and Asin-Cardiel E (2000) Electromagnetic therapeutic angiogenesis the next step. Neurol Res 22(4):349-50.

Edelberg J M et al. (2002) Platelet-derived growth factor-AB limits the extent of myocardial infarction in a rat model: feasibility of restoring impaired angiogenic capacity in the aging heart. Circultion 105(5):608-13.

Freedman S B and Isner J M (2002) Therapeutic angiogenesis for coronary artery disease. Ann Intern Med 136(1):54-71.

Symes J F (2000) Focal angiogenic therapy for myocardial ischaemia. J Card Surg 15(4):283-90.

Chou E et al. (2002) Decreased cardiac expression of vascular endothelial growth factor and its receptors in insulin-resistant and diabetic states: a possible explanation for impaired collateral formation in cardiac tissue. Circulation 105(3):373-9.

Hartlapp I et al. (2001) Fibrocytes induce an angiogenic phenotype in cultured endothelial cells and promote angiogenesis in-vivo. FASEB J 15(12):2215-24.

Epstein S E et al. (2001) Therapeutic interventions for enhancing collateral development by administration of growth factors: basic principles, early results and potential hazards. Cardiovasc Res 49(3):532-42.

### BRIEF DESCRIPTION OF THE PHOTOGRAPHS

FIG. 1(a). Structure of the essential amino acid Lysine. L- and D-enantomers are different by having mirror image orientation of the same groups and atoms around the chiral carbon atom. Note the two amino groups at the two ends of the molecule which probably gets protonated in situations of ischaemia (where tissue hydrogen ion concentration goes high to various levels depending on the degree of ischaemia) and possibly act as the binding sites for the growth factor(s)/angiogenic factor(s) on one end and to the receptor(s) on the other end. There is a concentration window for the molecule(s) to act as the molecular bridge.

(b) Oligo-lysine (MW˜1000) structure modeling in a non-aqueous minimum energy environment showing the amino groups projected at either ends “in a pack” (the possible binding sites, as explained above). Enhanced activity of the lysine/activated lysine molecule is mainly due to the conformational distribution of amino groups/protonated amino groups in space as may be evidenced from the above figure.

(c) Oligo-lysine (MW˜4500) structure model in a non-aqueous minimum energy environment showing disarrayed amino groups being projected from different directions in a non-coherent manner. The molecule was not effective in bringing about the cellular expansion/angiogenic response as in (b) above.

FIG. 2. (a) 3-D scanning electron microscopic photograph of spongy granulation tissue in experimental wound (acute surgical incision dressed with topically applied lysine) 72 hrs post surgery. (b) & (c) Close up views of granulation tissue of experimental acute wound as in (a). (d) Control wound (dressed with surface antibiotic but without lysine). Note the near total absence of spongy granulation tissue in the control.

FIG. 3. (a) 3-D scanning electron microscopic photograph of acut surgical wound treated with systemic lysine (i.p. injection, 5 ml inj., 5 mg/ml lysine soln. Note the spongy granulation tissue in the vicinity of the incision. (b) Control incision wound not treated with the systemic lysine solution as in (a). Note the total absence of the granulation tissue in the control wound as compared to the experimental wound (a). (c) Stratified layers of dividing cells, in-vivo, in acute surgical incision model following systemic application of the essential amino acid as described in (a) above.

FIG. 4. Histopathology of experimental and control acute surgical wounds in rabbit model. Note extensive cellular thickening from the basal keratinocyte layer in the experimental wound (a) compared to nearly no response in the control wound (b). Extensive angiogenic response was also obvious in the experimental wounds of human origin along with very advanced degree of Keratinocyte layer thickening.

FIG. 5. Example of an extremely protracted human chronic wound (bed sore of ˜8 weeks' duration. The wound was originally treated with conventional medication and regimen as described in the materials and methods section. Subsequently the patient was recruited for lysine therapy in the topical application category. Extensive granulation tissue and profuse angiogenic response was observed in the wound, as is obvious in the figure, following ˜two weeks' of topical lysine application (a, b) (please see the Example section). Extremely protracted (chronic) human stump wound treated with conventional medication and protocol (c) showed extensive granulation within two weeks of topical lysine therapy (d). The patient was taken for skin graft around two weeks (clinical end point).

FIG. 6. Example of Cellulitic wound following 3^{rd} degree burn in the hand. The wound was treated for 45 days with conventional medication and schedule as described in the material and method section. Conventional medication and therapy did not give rise to granulation tissue at all. Within third day of initiation of topical lysine therapy reasonable granulation/angiogenesis was obvious (a). On 11^{th} day post initiation of therapy, extensive granulation/angiogenic tissue was obvious in the wound bed (b, c). The patient was taken for skin graft around two weeks.

FIG. 7. High-lysine/derivative(s) formulation induced angiogenesis and granulation tissue formation in extensive burn case (2nd degree). Comparative condition in control subject included in the study showed near total absence of granulation in the wound bed by the time experimental wounds treated with the said formulation were nearing clinical end-point (˜2-3 weeks, depending on the condition including level of superadded infections, if any). During lysine therapy the experimental wounds did not receive any protective antibiotic coverage along the entire period of treatment. The burn case was included in the lysine therapy following extensive treatment with conventional modes of medications and regimen. Please note absolutely normal healing with epithelialization without any contracture, deformation, fibrosis with lysine therapy (which is so very common with all types of burn wounds, particularly in adults)

FIG. 8. Extensive burn wounds treated with high-lysine/derivative(s) formulation showed extensive angiogenic response very early in therapy as was described in FIG. 7 above. The experimental wounds did not receive any simultaneous therapy with any other angiogenic/granulation tissue inducing agent(s) neither any protective surface antibiotic preparation was used during the entire period of therapy).

FIG. 9. High-lysine/derivative(s) formulation alone (and without any other simultaneous medication including use of any protective surface antibiotic) gave rise to extensive angiogenic response very early in the treatment regime of protracted diabetic wound(s) and ulcer(s), compared to matched controls, both in terms of extent of angiogenic response as well as time scale. No simultaneous surface or systemic antibiotic coverage was employed for the experimental wounds during the entire period of therapy. This diabetic wound, which constitutes only a representative case, was included in the high-lysine formulation therapy after 12 weeks' of conventional therapy with conventional medications, which lead to formation of a extensive pouch-type ulcer around knee-joint.

FIG. 10. Histopathological photograph of healthy glandular structures in the experimental wounds (treated with topical lysine therapy) of human origin (chronic diabetic ulcer of long standing) (a,b,c). Note the extensive blood cell intravasation in the wound area around 11^{th} day post initiation of therapy, a clear indicator of regeneration of new hierarchical blood vessels (capillary neogenesis; angiogenesis) without simultaneous application of any other angiogenic/vasculogenic/granulation tissue inducing agent(s), nor any systemic and/or local antibiotic preparation(s) was ever used in the experimental wounds during the entire period of therapy.

FIG. 11. Histopathological representations of chronic human wounds treated with topical high-lysine preparation(s), as described before, showed extensive angiogenic responses reproducibly as was evident (a,b,c,d). The control mode(s) of therapy, in sharp contrast, showed very passive capillary response which were all very leaky without any active endothelial response and signs of regeneration as was evident in the experimental wounds(s).

FIG. 12. Cell culture photographs of d-Lysine (˜10 mcg/ml of added load, in addition to the usual load of l-Lysine, whatever was there in the media as the metabolic requirement) mediated cellular expansion in culture. The d-enantiomer of the essential amino acid was equally effective (as compared to the l-variety), in expanding cells in-vitro as well as in-vivo. In-situ controlled and physiological expansion of constituent cells in a wound bed, including the endothelial cells and their mediation of normal angiogenic process, mediate only by lysine/derivative(s), without addition/intervention of any other additive(s), whatsoever, is the essence of present invention.

FIG. 13. Graph of poly-lysine (MW˜1000) mediated adherent cellular growth in-vitro. Oligo-lysines with MWs in this range was effective in expanding cells, both in-vitro and in-vivo, comparable to that of l- and d-isomers.

FIG. 14. Photographs of ischaemic cardiac muscle tissues with (a and b) and without (c and d) application of lysine solution (according to the Material and Method section description) 21 days post surgery. Note the extensive angiogenic response in lysine treated ischaemic myocardium compared to the control. Also note the hierarchical nature of the angiogenic response which is evident from the capillaries filled with blood cells.

### DETAILED DESCRIPTION OF PREFFERED EMBODIMENTS

Before the present invention is described, it is to be understood that this invention is not limited to particular methodologies (e.g. modes of administration) or specific compositions described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by the reference to disclose and describe the methods and/or materials in connection with which the publications are cited.

The publications discussed herein are provided solely for their disclosure prior to the filing date of the present application. Nothing herein is to be construed as an admission that the present invention is not entitled to antidate such publication by virtue of prior invention. Further, the dates of publication provided may be different from the actual publication dates which may need to be independently confirmed.

Definitions

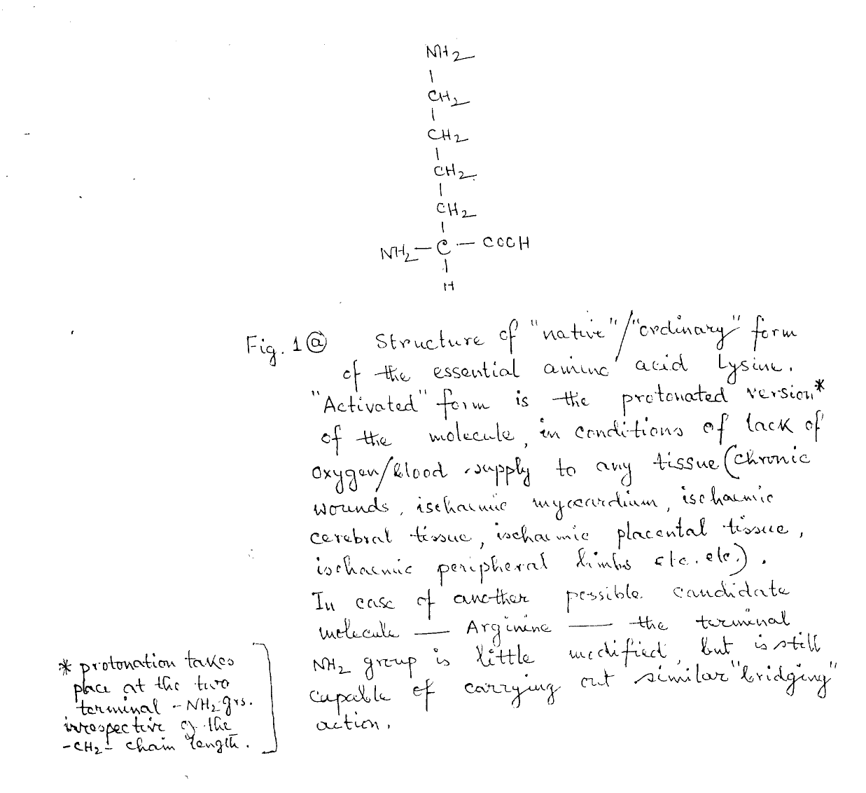

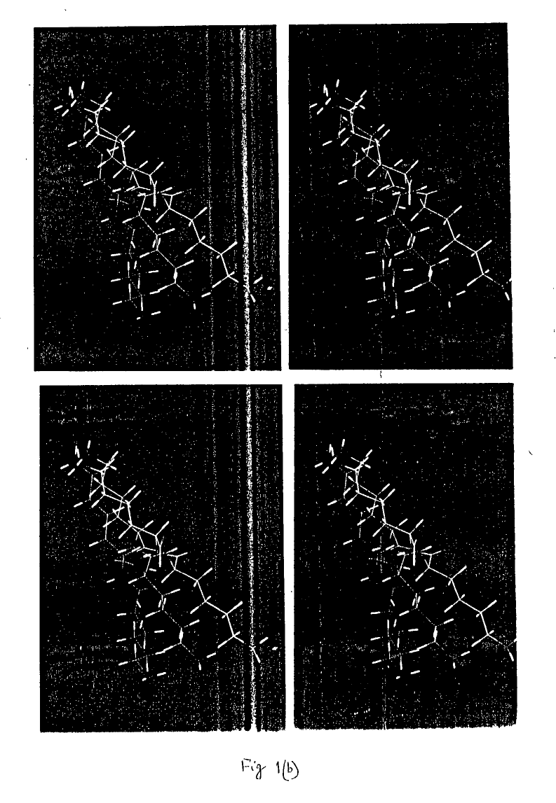

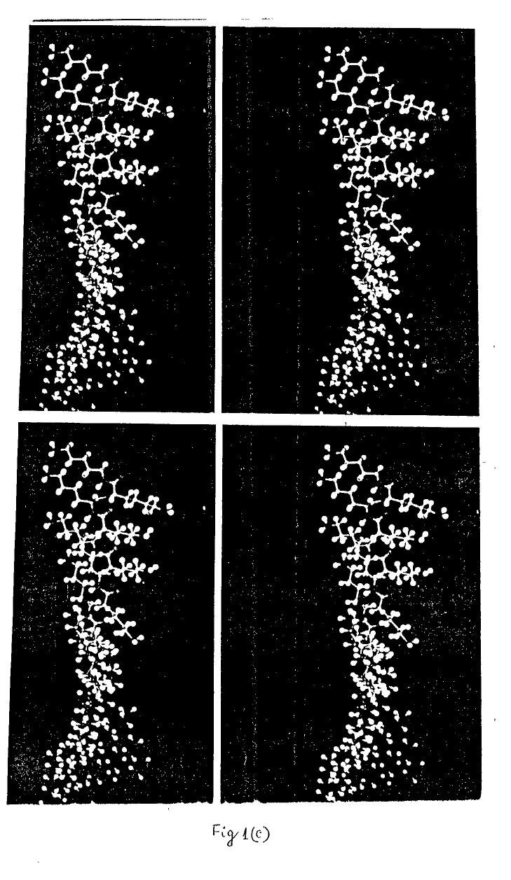

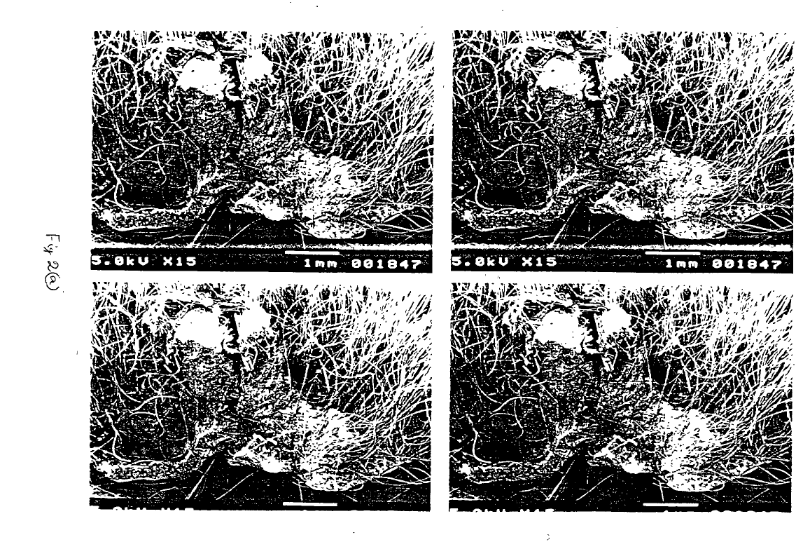

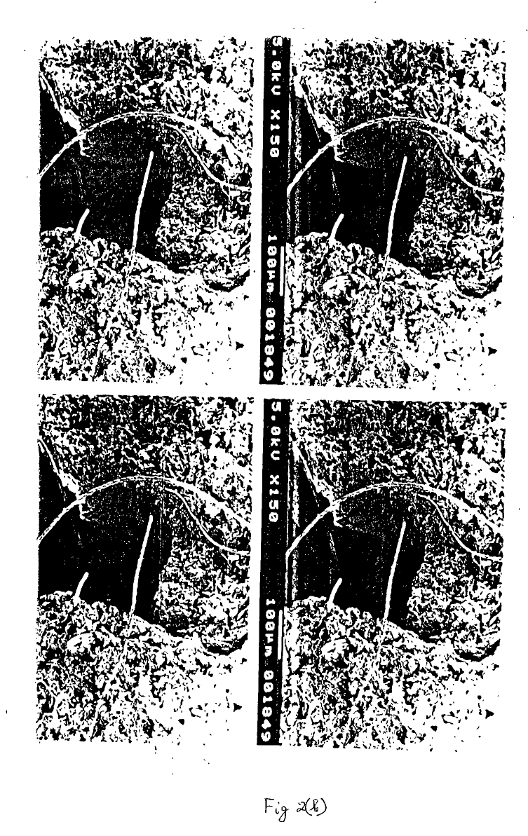

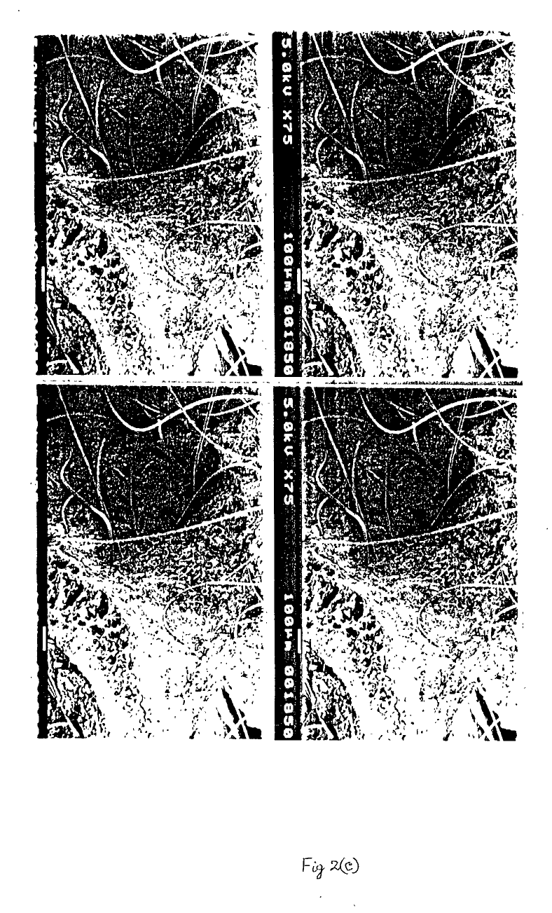

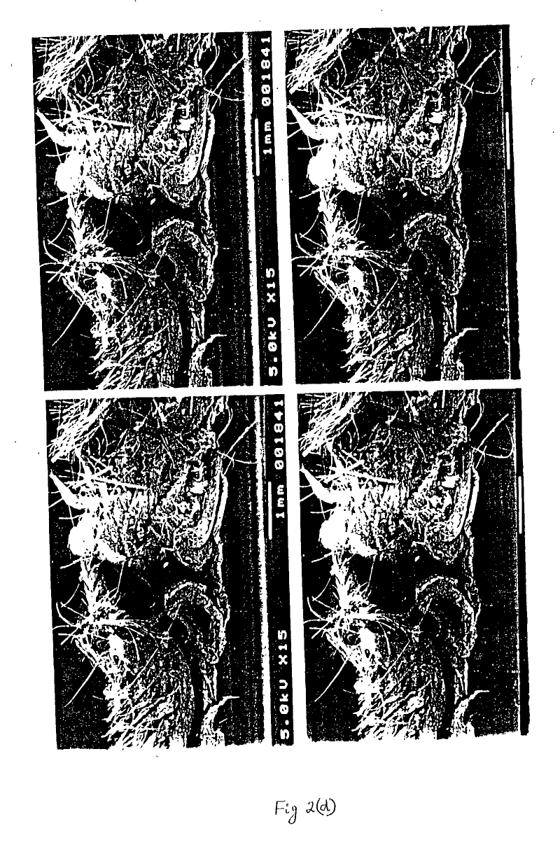

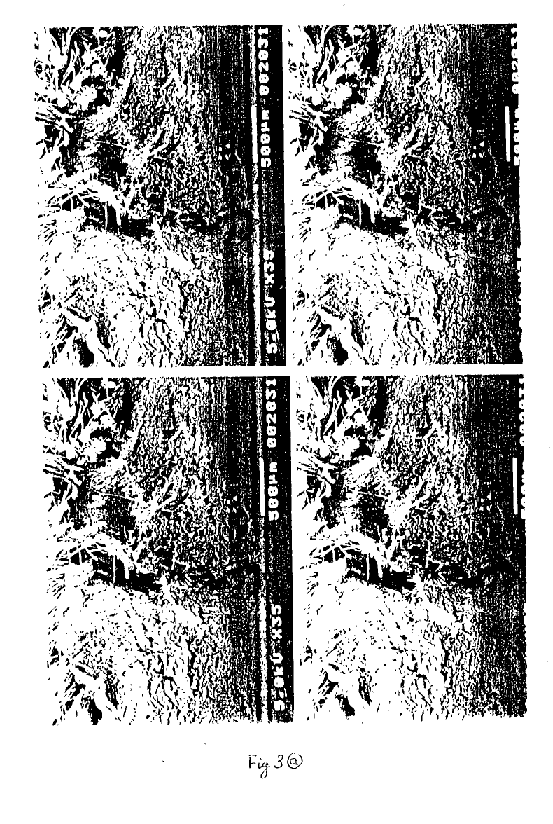

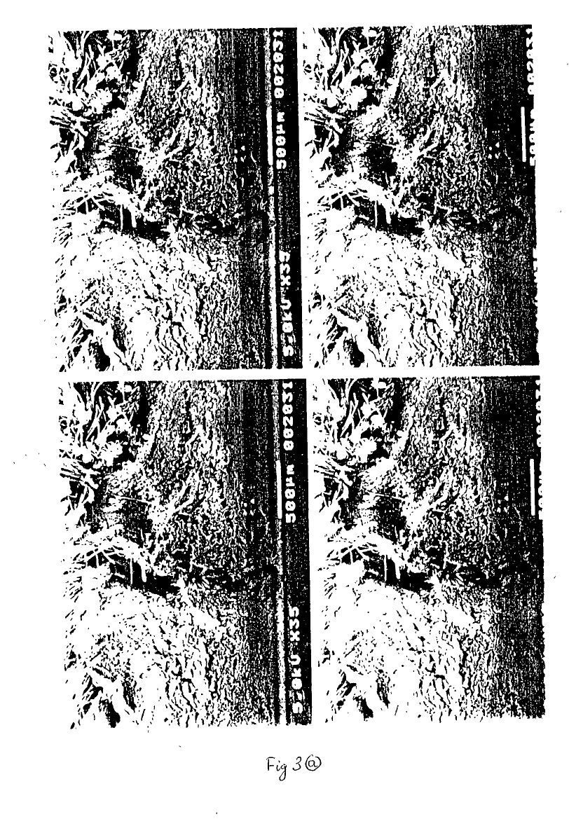

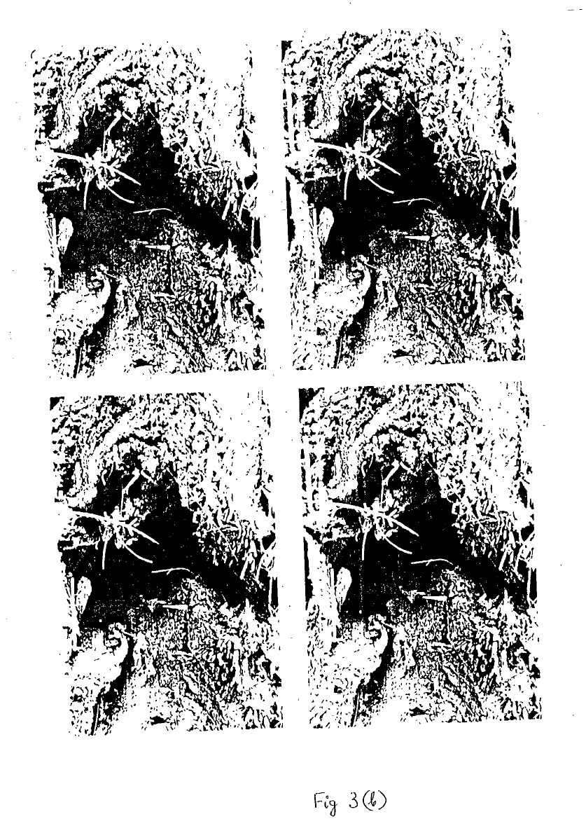

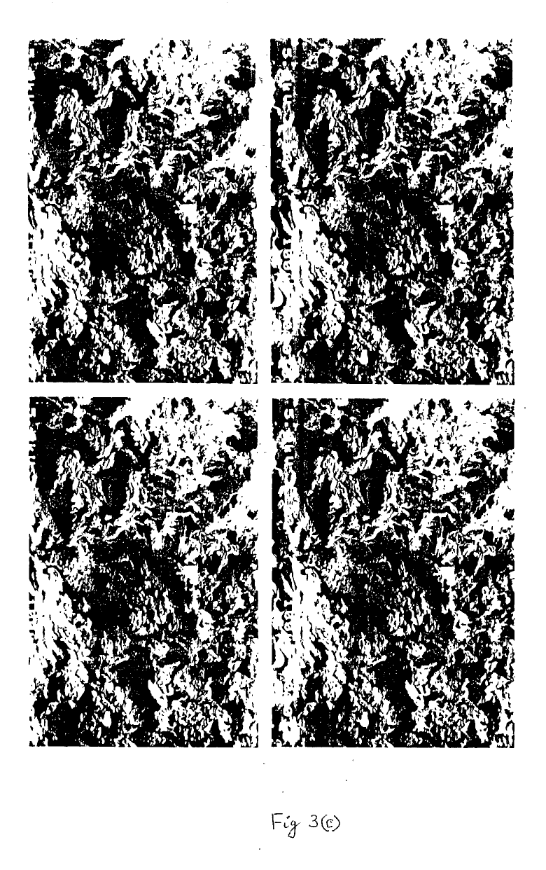

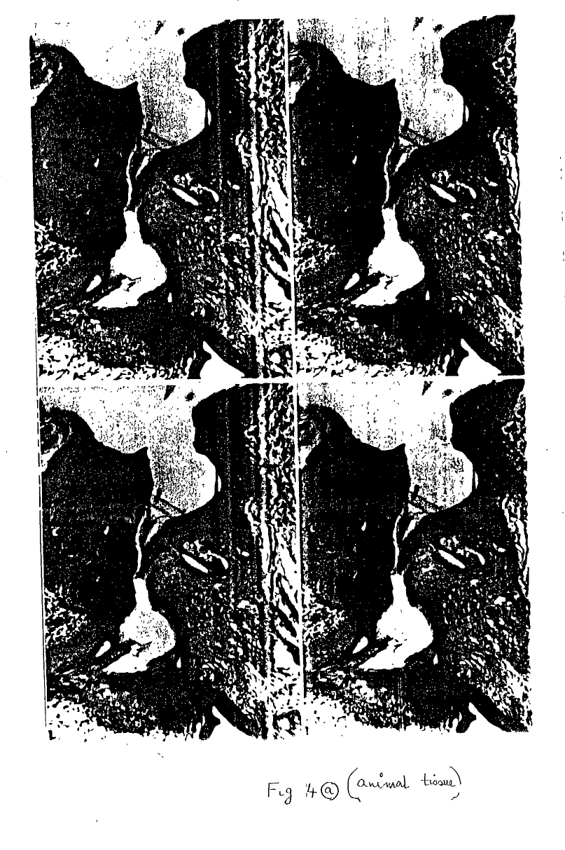

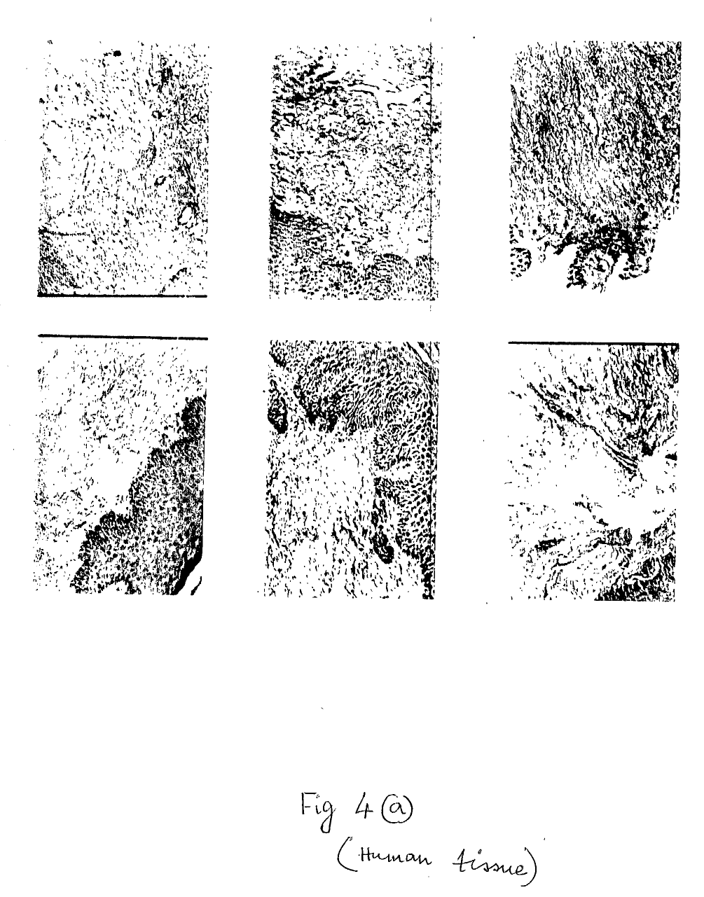

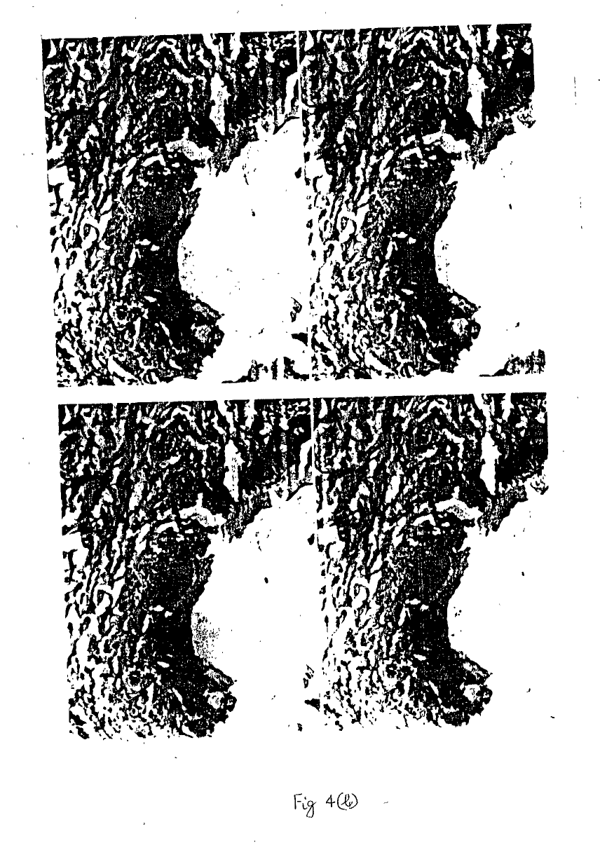

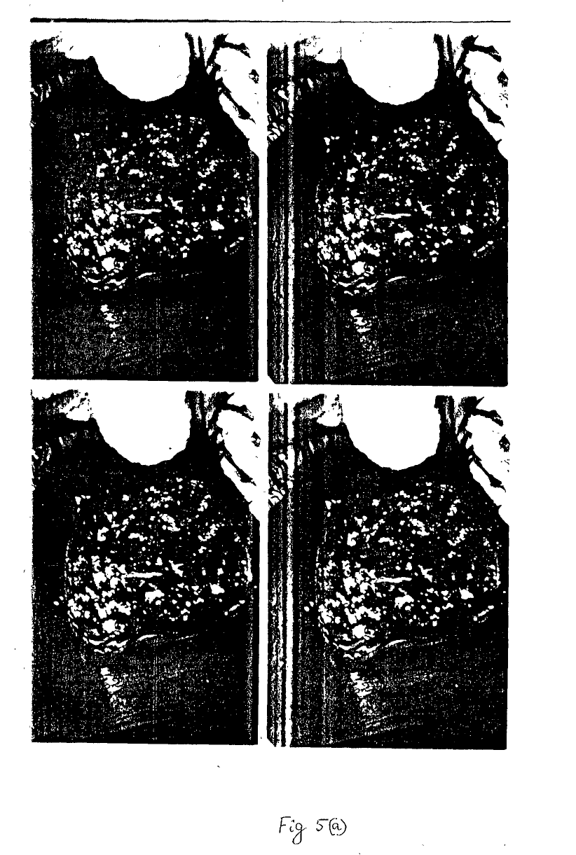

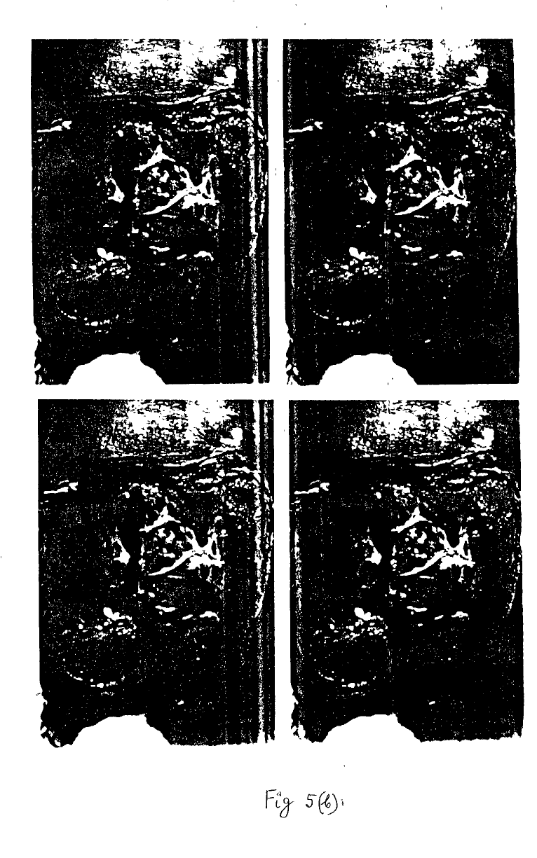

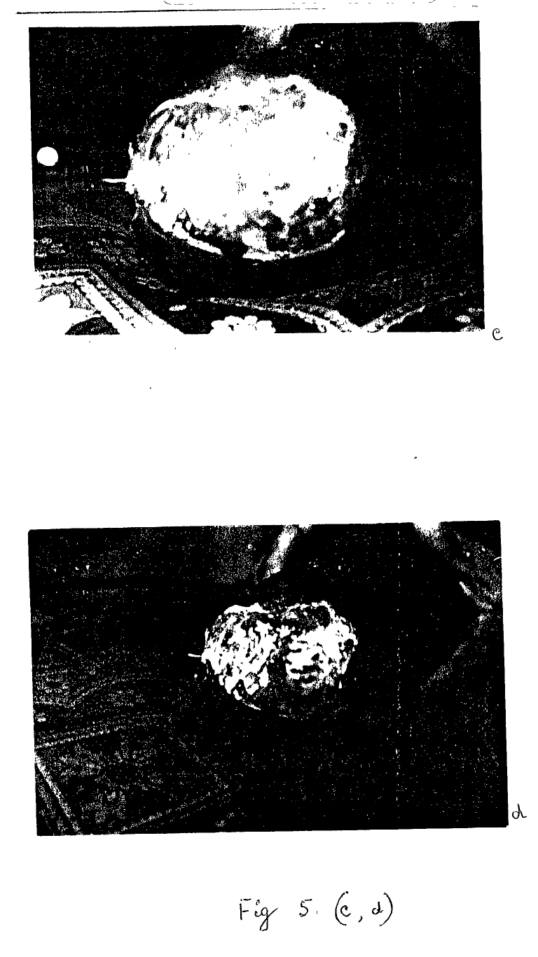

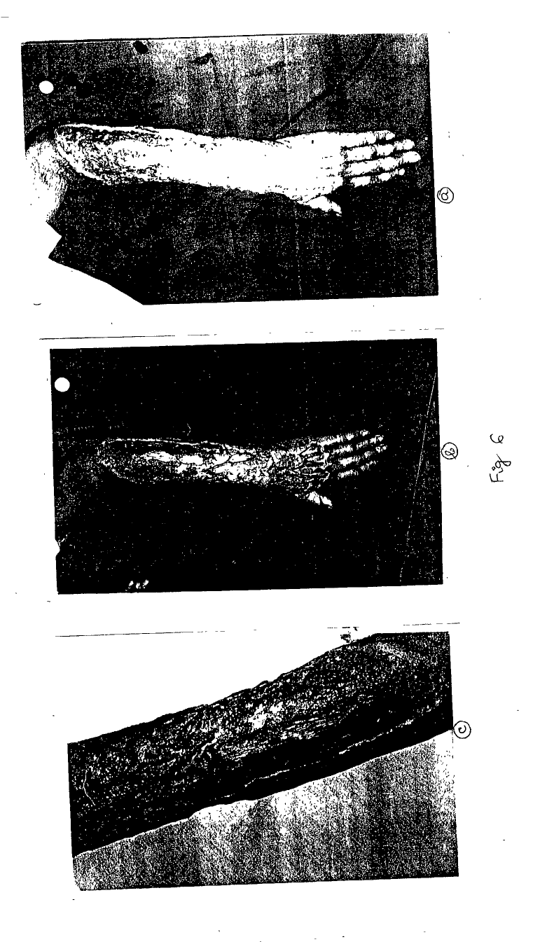

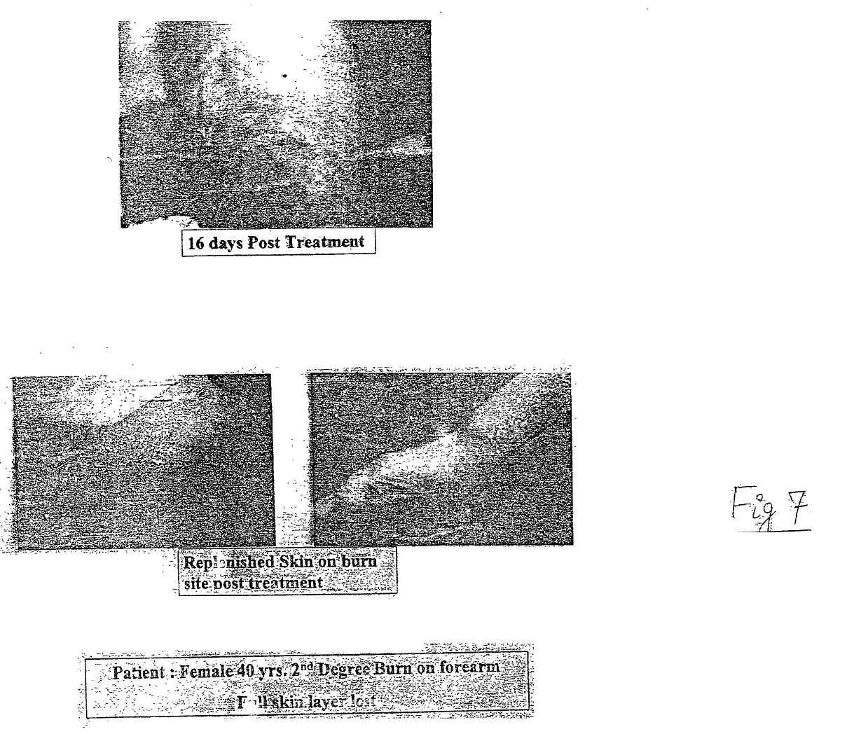

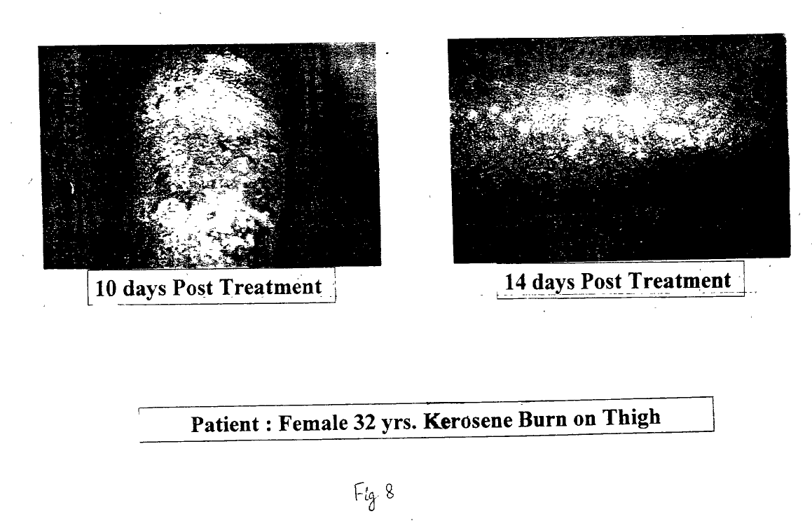

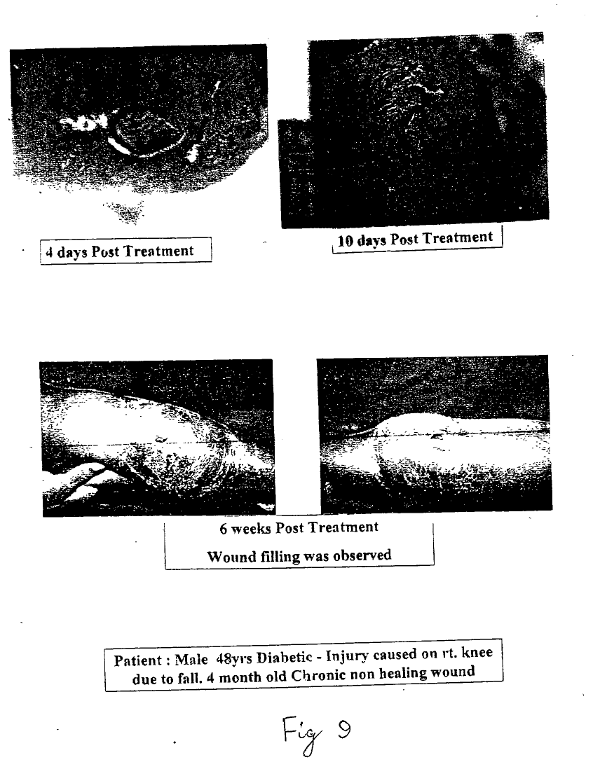

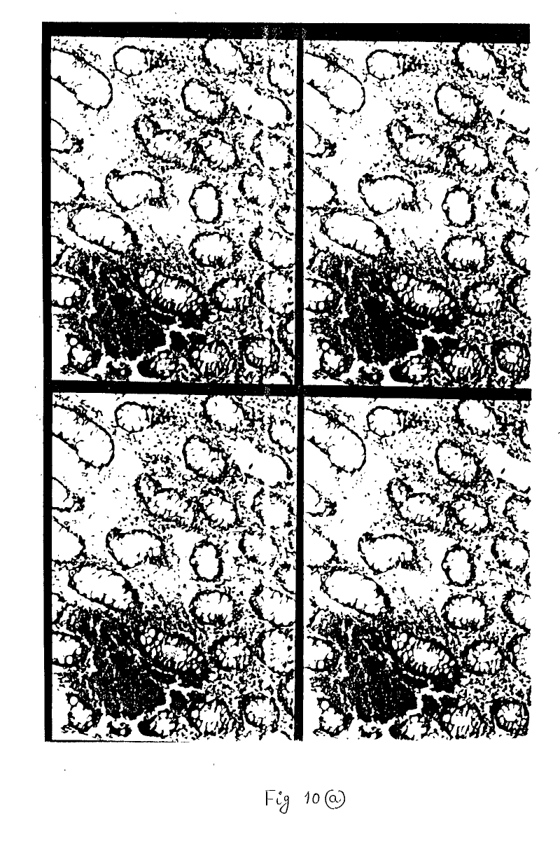

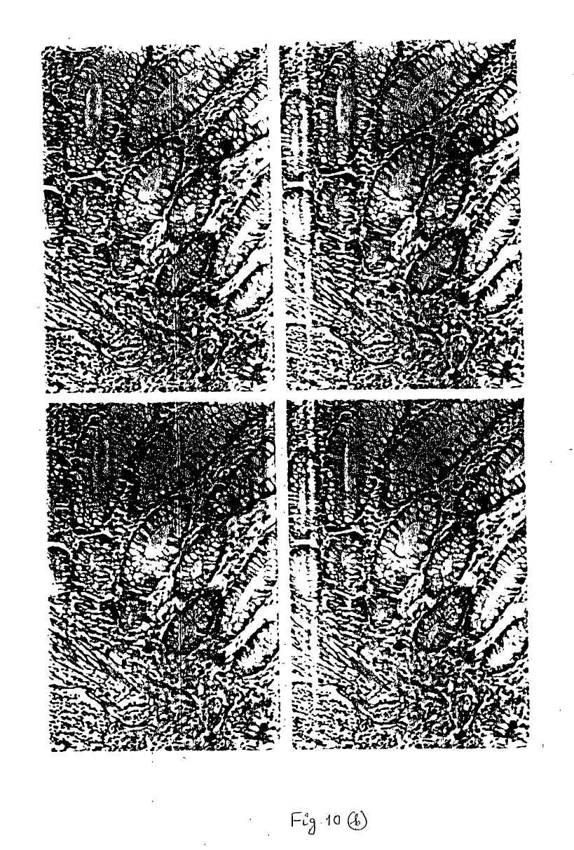

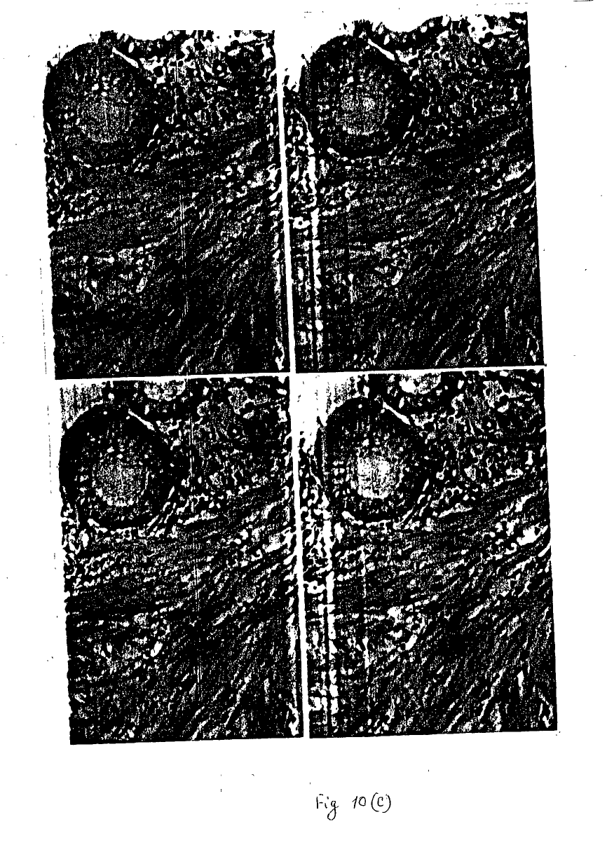

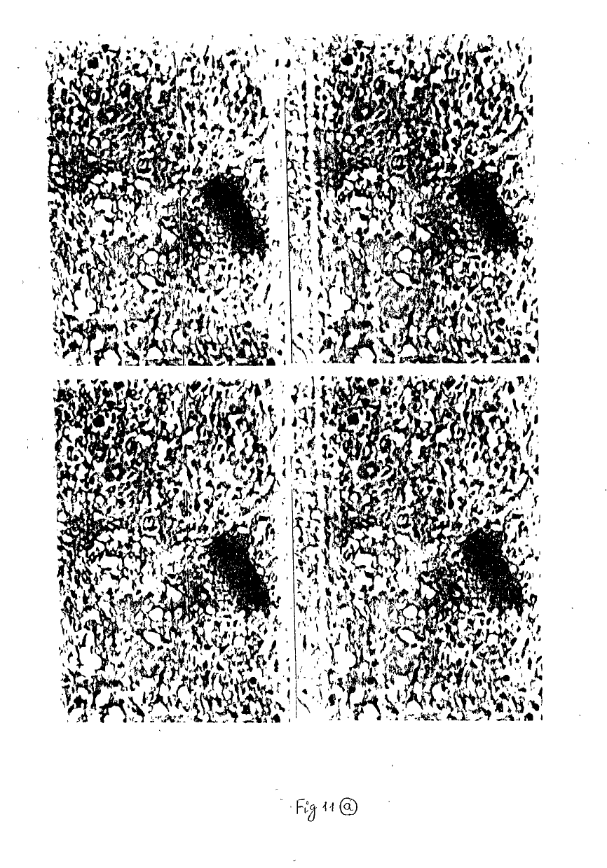

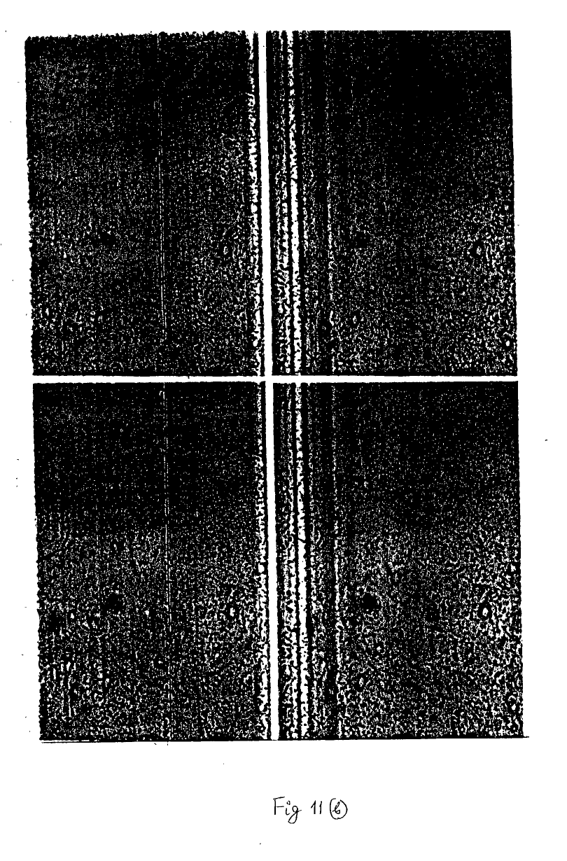

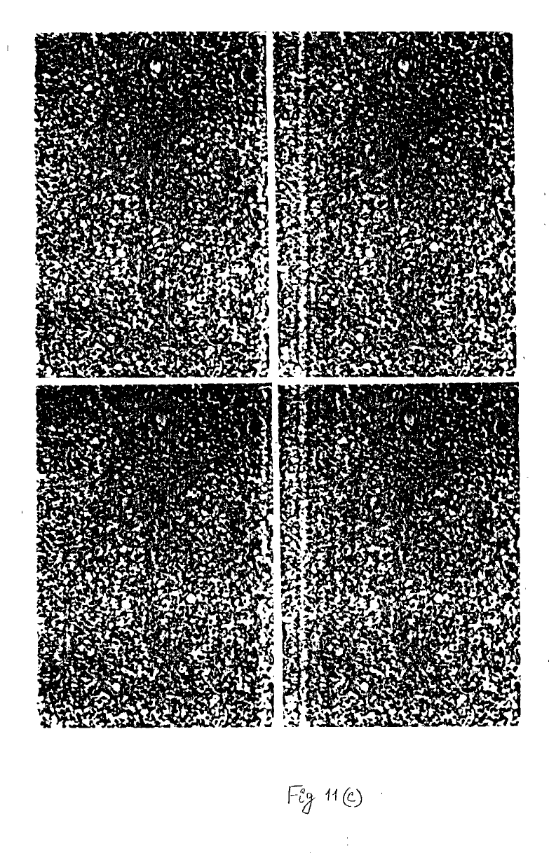

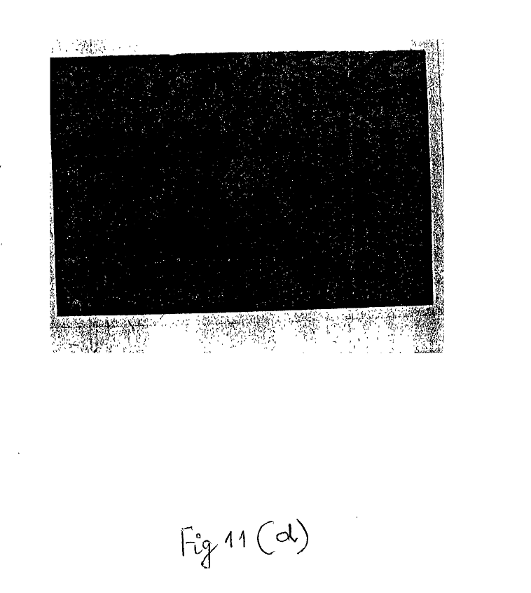

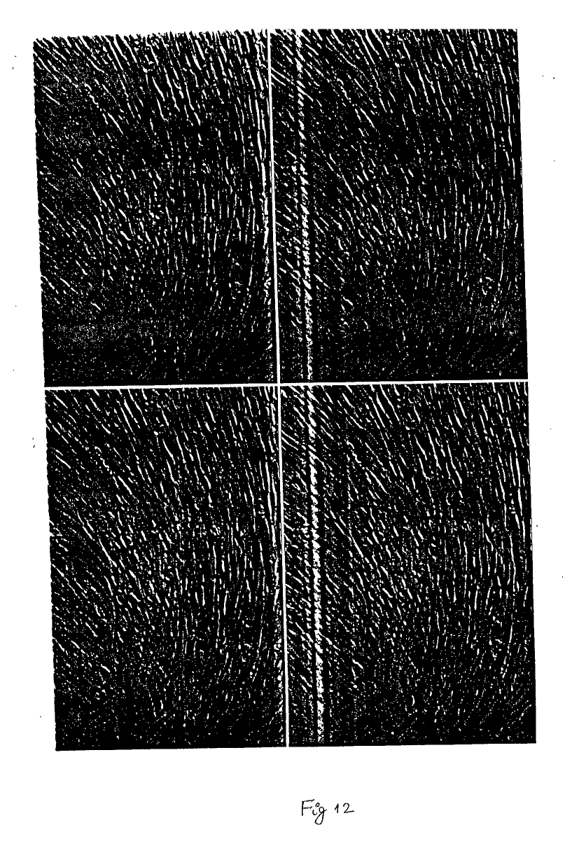

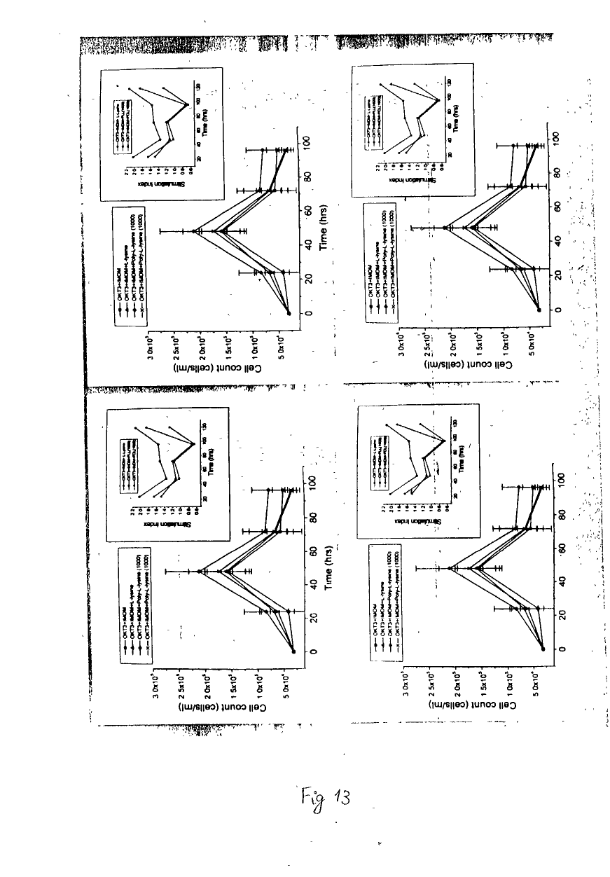

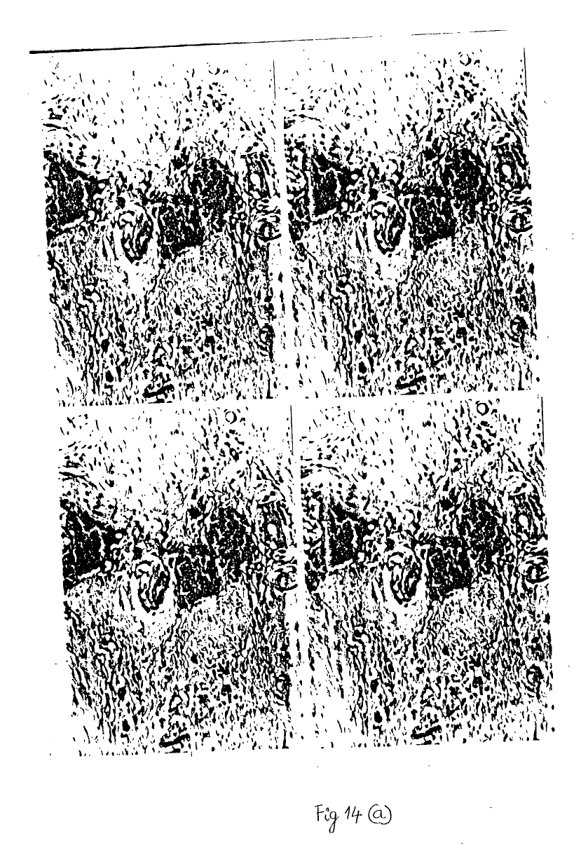

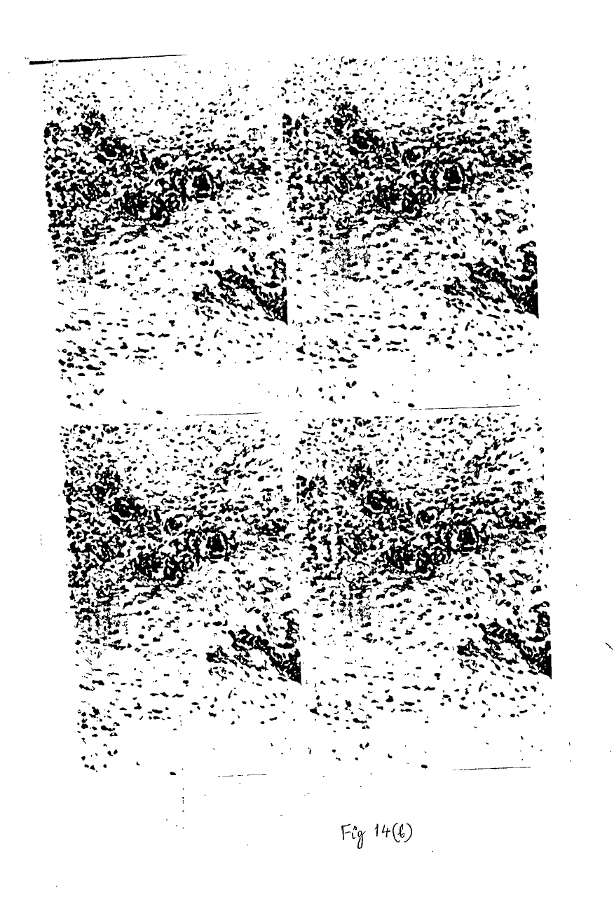

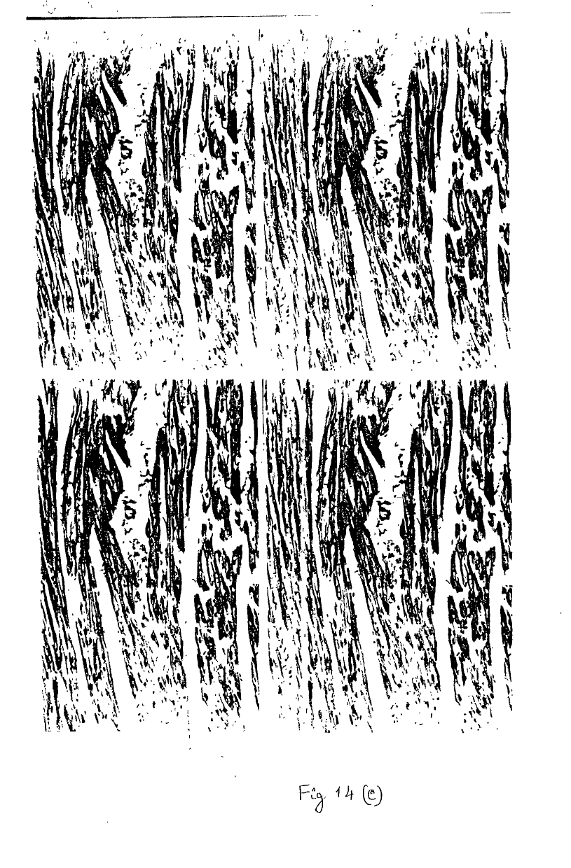

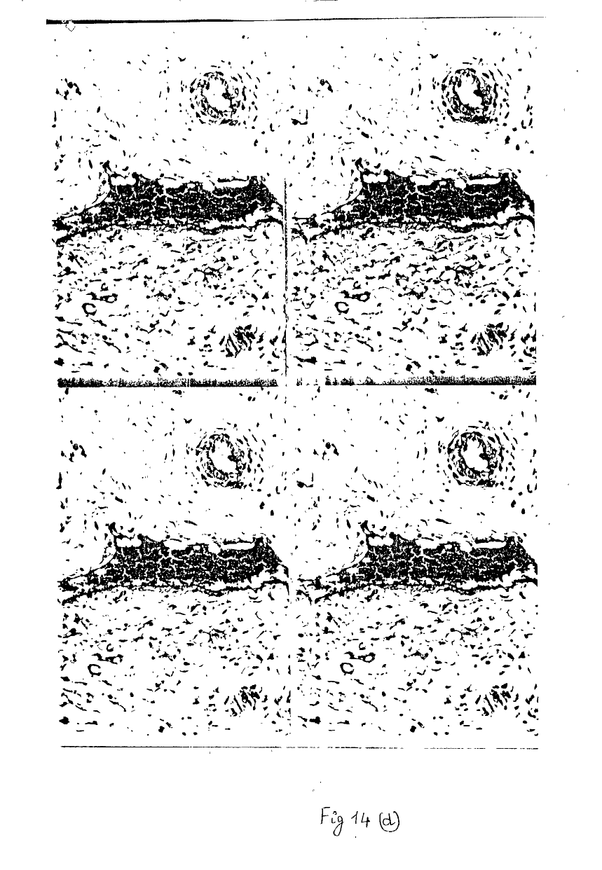

The term “Lysine” is intended to mean the naturally occurring cationic essential amino acid known as lysine and its d-isomer either in it's/their native form(s) or in “activated” form(s) where the terminal amino group(s), at each end of the molecule(s) is/are protonated in tissues suffering from lack of blood supply due to various reason(s)—the ischaemic tissue(s), which may be isolated and purified from nature or synthetically produced in any manner. This term is also intended to encompass the commonly occurring salts such as hydrochloride, hydrobromide, hydroiodide, nitrate, sulfate or bisulfate phosphate or acid phosphate, acetate, lactate, citrate or acid citrate, tartrate, bitartrate, succinate, maleate, fumerate, gluconate, saccharate, benzoate, methanesulfonate, ethanesulfonate, benzenesulfonate, p-toluene sulfonate, camphorate and pamoate salts. Lysine is an off-white/white, dry powder/partly granular/amorphous solid, having a mol. wt. of ˜150 and the formula shown in FIG. 1.

Unless specifically indicated otherwise, the term “Lysine” further includes any pharmacologically acceptable derivative or metabolite of lysie or structurally similar molecule(s) which exhibit(s) pharmacotherapeutic properties similar to lysine, including its oligomers (mol. wt. ˜500-2500). Such derivatives, metabolites and derivatives of metabolites and structurally homologous molecules are known in the art, and include, but are not necessarily limited to ornithinine, putrescine, cadaverine, arginine or pharamaceutically acceptable salts thereof.

The term (lysine) “derivatives” essentially means d-lysine and/or oligomers (up to or around M.W. ˜500 to 2500).

In this specification unless otherwise specified, the term “lysine” also encompasses within its ambit the compound in its “activated” form, accentuated or activated in-situ after application or administration predominantly under anaerobic conditions bringing about angiogenic responses.

The terms “treatment”, “treating” and the like are used herein to generally mean obtaining a desired pharmacologic and/or physiologic effect, e.g. stimulation of angiogenesis. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: a) preventing a disease or condition (e.g. preventing the loss of a skin graft or a reattached limb due to inadequate vascularization) from occurring in a subject who may be predisposed to the disease but has not yet been diagnosed as having it; b) inhibiting the disease, e.g., arresting its development; or c) relieving the disease (e.g. enhancing the development of a “bio-bypass” around an obstructed vessel to improve blood flow to an organ). In the context of the present invention, stimulation of angiogenesis is employed for subject having a disease or condition amenable to tretment by increasing vascularity and increasing blood flow.

By “therapeutically effective amount of lysine” is meant an amount of lysine and/or derivative(s), effective to facilitate a desired therapeutic effect, e.g., a desired level of angiogenic and/or vasculogenic stimulation. The precise desired therapeutic effect will vary according to the condition to be treated.

### OVERVIEW OF THE INVENTION

The present invention is based on the surprising discovery that lysine **only** (and without the presence of any other angiogenic factor(s)/angiogenic growth factor(s)/vasculogenic/granulation tissue inducing agent(s), as observed in all currently available formulations/preparations etc.), induces angiogenesis, in ischaemic tissues, on its own, even without the intervention of any other surface/systemic antibiotic(s), (indicated otherwise for the wounds in question, depending on the extent and severity), being added from outside. The inventor's initial inquiries were directed towards finding new chemical additives in a high-density-culture system, in-vitro, where the objective was to expand viable hybridoma cell mass to the maximum possible at the least possible time (lag phase minimization) and maintaining the viable cell mass in culture for the maximum possible time period, in both fed-batch as well as batch) cultures. The final goal was to get highest possible yields of monoclonal antibody (Mab) in the said high-density culture. Monomeric lysine was observed to expand the biomass (cell population) at the least possible time compared to other physico-chemical means (Datta, D et al. 1997). Because of its ability to expand cells in-vitro, the cationic amino acid was taken, along with its d-isomer and short oligomer (m.w. up to or around 500-2500) to in-vivo experimentation and clinical conditions, for controlled regeneration of cells (in clinical conditions), where in-situ regeneration of cells was centrally important along with angio-neogenesis/vasculogensis. Because the molecule has been proposed to have an indirect cell surface bridging role(s), between the cell surface receptors and the growth factor(s) (including the angiogenic factors), derived from the circulating serum protein pool, cellular expansion, migration (of the endothelial cells, forming the very early angiogenic buds) was found to be extremely rapid, reproducible and controlled (Datta, D, Unpublished results).

Thus the inventor has discovered that lysine, a cationic amino acid, provides the basis of a new therapeutic approach to enhance angiogenesis in the treatment of coronary, cerebral, peripheral or other occlusive arterial diseases; and for the enhancement of wound healing and the improved vascularization of surgically transplanted tissues or organs (e.g. skin graft or reattached limbs). In view of its remarkable angiogenic potency relative to other conventional angiogenic agents,

Lysine has significant advantage over the current candidates as the basis of therapeutic angiogenesis. Moreover the pharmacology and pharmacokinetics of the essential amino acid has already been well documented and methods for systemic as well as local delivery have been investigated. Processes for the manufacture of lysine and lysine oligomers are also well characterized. Furthermore, these small molecules, particularly the l-variety (l-lysine) is synthesized easily, has no shelf-life problem, extremely stable, highly biocompatible with no known toxicity/side effects even in very high loading doses and is least costly compared to all the other currently known angiogenic agents/factors (these are all mostly proteins or peptides in native).

Accordingly the invention encompasses methods and compositions for stimulation of angiogenesis and/or vasculogenesis by administration of the essential amino acid, its isomer and oligomer. Of particular interest is the stimulation of angiogenesis and/or vasculogenesis in-vivo, to effect increase in blood flow, increased capillary density, and/or increased vascularity within, adjacent, or around an ischaemic site.

Lysine, Lysine Isomer and Lysine Oligomer

The methods of the invention to stimulate angiogenesis are accomplished by administration of a cationic amino acid, particularly lysine, lysine isomer and oligomer. Methods for production of the essential amino acid and derivatives, as mentioned, are well known in the art.

Additional bi-amino compounds, similar structurally to the cationic amino acid in question, include but are not necessarily limited to ornithine, putrescine, cadaverine, arginine, which can be provided in a herbal preparation, either in isolated form (e.g. separated or partially separated from the materials that naturally accompany the said compounds).

Further biamino compounds of interest, analogous to the cationic amino acid in question, can be readily identified by the ability of the candidate molecule(s) or their combinations, to stimulate angiogenesis/vasculogenesis in-vivo (e.g. in acute surgical wound model in animal, in chronic human wound model, in ischaemic myocardium etc.)

Pharmaceutical Compositions

Upon reading the present specification, the ordinarily skilled artisan will appreciate that the pharmaceutical compositions comprising lysine and derivatives described herein can be provided in a wide variety of formulations. More particularly, the cationic amino acid can be formulated into pharmaceutical compositions by combination with appropriate pharmaceutically acceptable carriers or diluents and may be formulated into preparations in solid, semi-solid (e.g. gel), liquid or gaseous forms, such as tablet, capsules, powders, granules, ointments, solutions, suppositories, injections, inhalants and aerosols. The amino acid, being a naturally-occuring compound the pharmaceutical composition can also be provided as a herbal preparation.

The amino acid and/or its isomer/oligomer formulation used will vary according to the condition or disease to be treated, the route of administration, the amount of the active ingradient(s) to be administered, and other variables that will be readily appreciated by the ordinarily skilled artisan. In general and as discussed in more detail below, administration of the essential amino acid/derivative(s) or formulation can be either systemic or local, and can be achieved in various ways, including but not necessarily limited to, administration by a route that is parenteral, intravenous, intra-arterial, intrapericardial, intramuscular, intraperitoneal, transdermal, transcutaneous, subdermal, intradermal, oral and intrapulmonary, etc.

In pharmaceutical dosage forms, the cationic amino acid and its derivatives, isomers may be administered in the form of their pharmaceutically acceptable salts or they may also be used alone or in appropriate association, as well as in combination, with other pharmaceutically active compounds. The following methods and excipients are merely exemplary and are in no way limiting.

The amino acid/derivative(s) can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol, and if desired with conventional additives such as solubilizers, isotonic agents, suspending agents, emulsifying agents, stabilizers and preservatives.

Formulations suitable for topical, transcutaneous, and transdermal administration e.g. to administer the amino acid/derivative directly to a wound, may be similarly prepared through use of appropriate suspending agents, solubilizers, thickening agents stabilizers and preservatives. Topical formulations may also be utilized with a means to provide continuous administration of the amino acid/derivative(s) by, for example, incorporation into slow release pellets or controlled-release patches.

The amino acid/derivative can also be formulated in a biocompatible gel, which gel can be applied topically (e.g. to facilitate wound healing) or implanted (e.g. to provide for sustained release of the amino acid/derivative at an internal treatment site).

For oral preparations the amino acid/derivative(s) can be used alone or in combination with appropriate additives to make tablets, powders, granules, capsules for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired with diluents, buffering agents, moistening agents, preservatives and flavouring agents.

The amino acid/derivatives can be utilized in aerosol formulation to be administered via inhalation. The compounds of the present invention can be formulated into pressurized acceptable propellants such as dichlorodifluoromethane, propane, nitrogen and the like.

Furthermore, the amino acid and/or derivative(s) can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs and suspensions may be provided wherein each dosage unit, for example, teaspoonful, tablespoonful, tablet or suppository, contains a predetermined amount of the composition containing one or more active ingredients. Similarly, unit dosage forms for injection or intravenous administration may comprise the amino acid derivative(s) in a composition as a solution in sterile water, normal saline or another pharmaceutically acceptable carrier.

The term “unit dosage form”, as used herein, refers to physically iscrete units suitable as unitary dosages for human and/or animal subjects, each unit containing a predetermined quantity of the amino acid/derivative(s) calculated in an amount sufficient to produce the desired angiogenic and/or vasculogenic effect in association with a pharmaceutically acceptable diluent, carrier or vehicle. The specifications for the unit dosage forms of the present invention depend on the prticular compound employed and the effect to be achieved, and the pharmacodynamics associated with each compound in the host.

The pharmaceutically acceptable exipients such as vehicles, adjuvants, carriers or diluents are readily available to the public. Moreover pharmaceutically acceptable auxiliary substances, such as pH adjusting and buffering agents, tonicity adjusting agents, stabilizers, wetting agents & the like, are readily available to the public.

In addition to the cationic amino acid and/or derivative(s), the pharmaceutical formulations according to the invention can comprise or be administered in parallel with agents that enhance angiogenesis by enhancing nitric oxide (NO) levels or prostacyclin levels.

Alternatively or in addition the pharmaceutical compositions according to the invention can comprise additional angiogenesis inducing and/or vasculogenesis-inducing agents that act through other independent pathways (e.g. VEGF, FGF-a and FGF-b etc.).

Particularly where the aminoacid/derivative(s) are to be delivered for local applications, e.g. by intramuscular route, it may be desireable to provide the active ingradient(s) in a gel or matrix that can support angiogenesis, e.g. migration and proliferation of vascular cells into the matrix with endothelial tube formation. The gel or matrix can thus provide at least the initial substrate upon which new vessels form. For example, the gel or matrix can be extruded into an ischaemic region to form a path for new blood vessel formation so as to bypass an obstruction in the area.

Induction of Angiogenesis In-Vivo

In order to accomplish stimulation of angiogenesis in vivo (e.g. as in the context of therapeutic angiogenesis), lysine and/or derivative(s) can be administered in any suitable form/manner, preferably with pharmaceutically acceptable carrier(s). One skilled in the art will readily appreciate that a variety of suitable methods of administering lysine and/or derivative(s), in the context of the present invention to a subject are available, and although, more than one route can be used to administer a particular compound a particular route can provide a more immediate, more effective and/or is associated with fewer side effects than another one or more routes. In general lysine and/or derivative(s) can be administered according to the method of the invention by, for example, a parenteral, intravenous, intra-arterial, intrapericardial, intramuscular, intraperitoneal, transdermal, transcutaneous, subdermal, intradermal or intrapulmonary route.

Local administration can be accomplished by direct injection (e.g. intramuscular injection) at the desired treatment site, by introduction of the amino acid/derivative(s) formulations intravenously at a site near a desired treatment site (e.g. into a vessel or capillary that feeds a treatment site), by intra-arterial or intra-pericardial introduction, by introduction (e g. by injection or other method of implantation) of lysine/derivative(s) formulation(s) in a biocompatible gel or capsule within or adjacent to a treatment site, by injection directly into muscle or other tissue in which increased blood flow and/or increased vascularity is desired, by rectal introduction of the formulation(s) (e.g. in the form of suppository to, for example, facilitate vascularization of a surgically created anastomosis after resection of a piece of bowel etc.

In one particular application of interest, the lysine/derivative(s) formulation is employed in a bio-bypass method, wherein instead of performing a more invasive procedure, such as a coronary bypass operation, lysine/derivative(s) formulation(s) is administered to induce growth of new blood vessels around the blocked region. In this embodiment, the lysine/derivative(s) formulation can be administered in the area of and/or proximal to the ischaemic tissue to stimulate angiogenesis.

In some embodiments it may be desirable to deliver the lysine/derivative(s) formulation directly to the wall of a vessel. One exemplary method of vessel-wall administration involves the use of a drug delivery catheter, particularly a drug delivery catheter comprising an inflatable balloon that can facilitate delivery to a vessel wall. Thus, in one embodiment the method of the invention comprises delivery of lysine and/or derivative(s) to a vessel wall by inflating a balloon catheter wherein the balloon comprises one or more lysine/derivative(s) formulation covering a substantial portion of the balloon. The lysine/derivative(s) formulation(s) is held in place against the vessel wall promoting adsorption through the vessel wall. In one example the catheter is a perfusion balloon catheter, which allows perfusion of blood through the catheter while holding the lysine/derivative(s) against the vessel walls for longer adsorption times. Examples of catheters suitable for lysine/derivative(s) application include drug delivery catheters disclosed in U.S. Pat. Nos. 5,558,642; 5,554,119; 5,591,129; and the like.

In another embodiment of interest, the lysine/derivative(s) formulation is delivered in the form of a biocompatible gel, which can be implanted (e g. by injection into or adjacent a treatment site, by extrusion into or adjacent a tissue to be treated etc.). Gel formulations comprising lysine/derivative(s) can be designed to facilitate local release of the amino acid/derivative(s) and other active agents for a sustained period (e.g. over a period of hours, days, weeks, etc.) The gel can be injected into or near a treatment site, e.g. using a needle or other delivery device. In one embodiment the gel is placed into or on an instrument which is inserted into the tissue and then slowly withdrawn to leave a track of gel, resulting in the stimulation of angiogenesis along the path made by the instrument. This latter method of delivery may be particularly desirable for the purpose of directing course of the bio-bypass.

In other embodiments it may be desirable to deliver the lysine/derivative(s) formultion(s) topically e.g. for localized delivery e.g. to facilitate wound healing. Topical application can be accomplished by use of a biocompatible gel, which may be provided in the form of a patch, or by use of a cream, foam and the like. Several gels, patches, creams, foams, and the like appropriate for application to wounds can be modified for delivery of lysine/derivative(s) formulation(s) according to the invention (see, e.g. U.S. Pat. Nos. 5,853,749; 5,844,013; 5,804,213; 5,770,229 etc.). In general topical administration is accomplished using a carrier such as a hydrophilic colloid or other material that provides a moist environment. Alternatively, for the purpose of wound healing the lysine/derivative(s) could be supplied, with or without other angiogenic agents in a gel or cream that could be applied to the wound. An example of such an application would be as a sodium carboxymethyl cellulose based topical gel containing the lysine/derivative(s) and other ingredients together with preservatives and stabilizers.

In other embodiments the lysine/derivative(s) formulation is delivered locally or systemically, preferably locally, using a transdermal patch. Several transdermal patches are well known in the art and such patches may be modified to provide for delivery of an amount of lysine/derivative(s), effective to stimulate angiogenesis according to the invention (see, e.g., U.S. Pat. Nos. 4,920,989; and 4,943,435 and the like).

In other methods of delivery, the lysine/derivative(s) can be administered using iontophoretic techniques. Methods and compositions for use in iontophoresis are well known in the art (see, e.g., U.S. Pat. Nos. 5,415,629; 5,899,876; 5,807,306; and the like).

The desirable extent of angiogenesis will depend on the particular condition or disease being treated, as well as the stability of the patient and possible side effects. In proper doses and with suitable administration, the present invention provides for a wide range of development of blood vessels e.g. from little development to essentially full development.

Lysine/derivative(s) based formulations' mediated stimulation of angiogenic response in tissues, where reperfusion is desired, is a phenomenon which is auto regulated in-vivo. The bi-amino cationic amino acid/derivative(s) exert it's angiogenic response by bridging the growth factors (angiogenic factors) to their cell surface receptors. Therefore, the entire process of enhancement of angiogenesis, according to the present invention is entirely dependent on the availability of the amino acid/derivative(s) in a relatively high concentration in the ischaemic zone/in its immediate vicinity, as well as on the presence/availability of naturally occurring angiogenic factor(s) in the zone of ischaemia/in the immediate vicinity.

Dose

The dose of lysine/derivative(s) administered to a subject, particularly a human, in the context of the present invention should be sufficient to effect a therapeutic angiogenic response in the subject over a reasonable time frame. The dose will be determined by the condition of the subject, as well as the body weight of the subject as well as the level of angiogenic growth factor(s) in an ischaemic zone/area of tissue, number of available receptor sites (for these angiogenic factors) at a given moment of ischaemia and the level of proton concentration ([H+]) (degree of ischaemia) in the ischaemic zone. The cationic amino acid being non-toxic, non-mutagenic, extremely biocompatible, loading doses have also been reported without any side effects/adverse effects.

In determining the effective amount of lysine/derivative(s) in the stimulation of angiogenesis, the route of administration, the kinetics of the release system (e.g. pill, gel or other matrix) and the potency of the active ingredient(s) is considered so as to achieve the desired angiogenic effect(s) with minimal adverse side effect(s). The amino acid formulation(s) will typically be administered to the subject being treted for a time period ranging from a day to few weeks, consistent with the clinical condition(s).

The following dosages assume that Lysine is being administered or a lysine derivative with similar potency and efficiency as lysine. As will be readily apparent to the ordinarily skilled artisan, the dosage is adjusted for lysine derivative(s) according to their potency and/or efficacy relative to lysine. If given orally, the dose may be in the range of 10 mg to 400 mg given 1 to 20 times daily and can be up to a total daily dose of about 10 mg to 8 gm. If applied topically, to provide a local angiogenic effect (mostly for wound repair applications), the dose would likely be in the range of about 0.001 mg to 10 mg per sq. cm. (wound) surface area. If injected for the purpose of a local effect, the matrix is designed to release locally an amount of cationic amino acid equivalent (l-, d-, and oligo-lysine is/are equally effective, on their own, in inducing angiogenic response) in the range of about 0.001 mg to 500 mg/unit area at the peak of activity. If injected for the purpose of a systemic effect, the matrix in which the lysine/derivative(s) is administered is designed to provide for a systemic delivery of a dose in the range of about 0.001 mg to 500 mg/dL of blood. If applied topically, for the purpose of a systemic effect, the patch or cream or gel (or any other suitable skin formulation) would be designed to provide for systemic delivery of a dose in the range of about 0.001 mg to 500 mg/dL of blood.

Regardless of the route of administration, the dose of lysine/derivative(s) can be administered over any appropriate time period e.g. over the course of 1 to 24 hrs, over 1 to several days, etc. Furthermore, multiple doses can be administered over a selected time period. A suitable dose can be administered in suitable sub-doses per day, particularly in a prophylactic regimen. The precise treatment level will be dependent upon the response of the subject being treated. In the treatment of some individuals with lysine/derivative(s), it may be desirable to utilize a megadosing regimen. In such a treatment, a large dose of the lysine/derivative(s) is administered to an individual, and with time the active ingredient(s) get eliminated through mainly two routes: a) rapid excretion through kidney, because of low mol. wt., and, b) rapidly taken up by he cells for their metabolic needs. For the d-isomer, the molecule gets eliminated quickly unaltered because of its non-utilization in physiological system and low mol. wt.

Conditions Amenable to Treatment by Lysine/Derivative(s)-Mediated Induction of Angiogenesis

The methods and lysine/derivative(s)-comprising compositions of the invention can be used to treat a variety of conditions that would benefit from stimulation of angiogenesis, stimulation of vasculogenesis, increased blood flow, and/or increased vascularity.

Examples of conditions and diseases amenable to treatment according to the method of the invention include any condition associated with obstruction of a blood vessel, e.g. obstruction of an artery, vein, or of a capillary system. Specific examples of such conditions or diseases include but are not necessarily limited to, coronary occlusive disease, carotid occlusive disease, arterial occlusive disease, peripheral artery disease, atherosclerosis, myointimal hyperplasia (e.g. due to vascular surgery or balloon angioplasty or vascular stenting), thromboangitis obliterans, thrombotic disorders, vasculitis and the like. Examples of conditions and diseases that can be prevented using the methods of the invention include but are not necessarily limited to stable or unstable angina, heart attack (myocardial infarction) or other vascular death, stroke, death or loss of limbs associated associated with decreased blood flow, and the like.

Other forms of therapeutic angiogenesis include, but are not necessarily limited to, the use of lysine/derivative(s) to accelerate healing of wounds or ulcer; to improve the vascularization of skin grafts or reattached limbs so as to preserve their function and viability; to improve the healing of surgical anastomosis (e.g. as in reconnecting portions of the bowel after gastrointestinal surgery); and to improve the growth of skin or hair.

### EXAMPLES

The following examples are put forth so as to provide those of ordinary skill in the art with a complete disclosure and description of how to make and use the present invention, and are not intended to limit the scope of what the inventor regards as his invention nor are they intended to represent that the experiments below are all or the only experiments performed. Efforts have been made to ensure accuracy with respect to numbers used (e.g. amounts, temperature etc.) but some experimental errors and deviations should be accounted for. Unless indicated otherwise, parts are parts by weight, wherever applicable, molecular wt. is weight average molecular weight temperature is in degrees Centigrade (wherever applicable) and pressure is at or near atmospheric (wherever applicable).

Materials and Methods

The following is a description of the methods and materials used in the specific examples below.

Animals

Rabbits (˜1.5 Kg body wt.) from Haffkine Biopharmaceuticals, Mumbai, India were used for acute surgical wound model experimentations. Two groups were maintained—experimental and control (n=5 in each)—for the study of “lysine”induced angiogenesis and granulation tissue formation in acute surgical wounds.

For cardiac angiogenesis experiments, healthy immunized adult sheep, weighing 20-30 Kgs were selected. The selected animals underwent routine blood tests and echocardiography prior to surgery which acted as the base line data. Under general anaesthesia, 4 sheep were operated through a left thoracotomy. A small area of infarction were created on the left ventricle by ligating one or two distal branches of coronary arteries (depending on the degree/number of collateral/interconnecting vessels as was observed in surgery. The infracted areas were identified on the table by the blanching of tissue and paradoxical movement of the infracted segment. Saturated solution of lysine at room temperature and pressure (1 ml) was injected intramuscularly in and around the zone of ischaemia in the experimental animals, whereas in the control animal normal saline (1 ml) was injected in place of lysine solution. In one of the experimental animals, in addition to the intramuscular injection of lysine, ˜10 ml of the saturated solution of lysine was injected at the root of the aorta through a pig-tail catheter passed retrogradely through the descending thoracic aorta. These injections were were made as a double blind study where the investigator did not know the sheep to which lysine solutions were injected intramuscularly. Post-operative echocardiogram were done in all the animals at 96 hrs post-surgery to assess the left ventricular wall motion. Two weeks post-surgery echocardiograms were done to assess left ventricular wall motion. The animals were sacrificed and autopsy of the hearts were done for examination of the degree of angiogenesis.

For acute surgical wound experiments, dorsal paravertebral skin was shaved in both the groups. Full thickness skin incisions (˜1 inch long) were made on either side of the vertebral column under local anaesthesia. In the experimental group, “lysine” in powder form(s) was instilled/spinkled/applied (˜10-15 mg) in each wound and the wounds were closed by single suture each to keep the edges apposed. No surface/systemic antibiotics were given to the animals dressed with “lysine”. Similar treatments were given to the control wounds except for omitting the “lysine” dressing altogether. Control wounds were protected with surface antibiotics ointment, applied at the time of each dressing. The wounds were covered with adhesive plasters in both the groups. Dressings were replaced every 48-72 hrs.

Human Subjects

Chronic wounds of different anatomical locations as well as etiology were included in the controlled open trial of “lysine” for it's/their ability to induce angiogenesis and induction of the healthy granulation tissue formation in these chronic wounds. These included diabetic wounds and ulcers of various durations and severity, bed sores and pressure sores of varios durations, infected wounds and cellulitic wounds of various origins, burns of different degrees and extents—both infected and otherwise, amputations, skingrafts, venous ulcers and even leprotic wounds and ulcers of long standing.

The wounds were debrided manually with gauge pieces soaked in normal saline to the point of bleeding of smaller vessels at the base of the wounds (wound-bed). Some of the chronic wounds were debrided surgically depending on the severity of the wound condition(s). Dressings were done with adequate amounts of the amino acid and/derivative(s) applied to the wound site(s) topically/locally. Wounds were closed with moistened gauge piece and adhesive tapes and the procedure repeated every 72-96 hrs. This approach constituted the experimental group subjects. In the control groups, conventional therapy were instituted both in terms of surgical/manual/enzymatic debridements as well as applying usual medications e.g. betadine, salutyl (a preparation which removes ischaemia by inducing angiogenesis in ischaemic wounds), trypsin, chymotrypsin, metronidazole preparations, silver sulfadiazine, regranex (a PDGF based healing agent containing “small” amount of the cationic amino acid lysine as “stabilizing agent”, along with quite a few other additives and ingredients), various surface antibiotics etc., depending on the clinical condition in question.

Histopathology

Tissue biopsies were taken from both groups—either animals or human beings—from appropriate wound site(s)/cardiac tissue (myocardium) at various time points post surgery/applications/dressings with the active ingredient(s), fixed at room temp. for 24 hrs. in formal saline (4% v/v). Tissue sections were stained with haematoxylin and eosin and observed under light microscope.

For examination of wound tissues under scanning electron microscope, skin tissues were taken (˜1.5 inch square) surgically under local anaesthesia in such a way that the original wound incision lied at the center of this piece of skin tissue, which was fixed in formal saline (4%) for 24 hrs at room temp. and pressure. Before mounting on to the observation stub with the help of a double faced adhesive tape, the wet tissues were dried adequately first with tissue papers and then at 37 degree Centrigrade incubator for ˜2 hrs. Finally the dried tissues were cut with scissors in such a way that the cut line in each case was perpendicular to the original incision line. The finally prepared samples were gold coated in a hummer for around 4 minutes in vacuum and examined in a scanning electron microscope (Jeol).

Cardiac tissues were processed for histopathology the same way as the skin tissue except for the fact that biopsies were taken 96 hrs post surgery and at the end of the experiment (2 weeks from the day of surgery).

### Example 1

### In Situ Tissue (Skin Wound) Repair Effect of Lysine

To study the effects of lysine, on, in-situ-tissue-repair, experiments were conducted as described in the method section above. Lysine alone and/or in combination with one or more component(s) of the derivative(s) as described in the “definition” was reproducibly observed to induce excellent tissue regeneration in-situ, in the form of healthy granulation tissue in all types of acute and chronic wounds of both animal and human origin. In case of chronic wound's, repair-by-in-situ-regeneration were observed to get initiated within the first 72-96 hours of institution of topical and/or systemic therapy (FIG. 2 and FIG. 3 and FIG. 4) with lysine in experimental wounds—both acute and chronic. This much faster rate of repair-by-regeneration was in sharp contrast to the conventional modes of therapy, the control wounds, which received the conventional approach of therapy as has been described in the method section above. In control wounds, receiving conventional therapy as described in the material & method section, appearance of granulation tissue was always delayed to 2-2½ weeks, by which time (only) lysine treated wounds in all categories reached clinical endpoints, as has been mentioned.

### Example 2

### Comparison of Repair Effects of Lysine with Other Preparations

As has been elaborated above in the material and method section, only-lysine treated wounds of different anatomical locations and origins and durations were compared with the similar wounds which were managed clinically with the conventional modes (formulations were as mentioned and schedule of treatment were as per convention, depending on the clinical state of the wounds being treated). As mentioned in Example 1, healthy granulation tissue was reproducibly observed to first appear in lysine treated wounds within 72-96 hrs as compared to control wounds (granulation tissue appeared around 2-2½ weeks or even beyond that depending on the condition of the wound, by which time experimental wounds, treated with lysine, reproducibly reached the clinical endpoint, of being ready for skin graft) (FIG. 4 and FIG. 5).

The following clinical conditions (wounds/ulcers) were also studied under the comparative scheme (only lysine treated wounds vs control wounds) as is described below:

Bed Sores

High-lysine formulation (preferably >10%, more preferably >30%, most preferably >75% and ideally >90% and/or up to 100% w/w) either in suitable and acceptable base or alone, was employed in dressing of bed sores (pressure sore) at 48-72 hrly intervals, without any other medication and without any surface/systemic antibiotic coverage (FIG. 5). Control wounds (bed sores) were treated with conventional medication as described, during the entire period of comparative study (all the control wounds subsequently, got included in the lysine-treated category after comparative study period, which was typically around 2 weeks from the date of institution of therapy. This was done to give the control patients the benefit of the finding, along with all the experimental patients. Represented case in FIG. 5 is one such, where the bed sore (˜16 inches×18 inches) was first treated for 42 days with conventional medication and regimen, as outlined in the method section. During this entire period, the wound was totally non-responsive to treatment and kept on increasing in size and was full of necrotic tissues (and discharges), remnants of which can still be seen in the corners. This particular example is included with the purpose of demonstrating the phenomenal degree of granulation tissue (angiogenic response) formation when the phenomenally low-cost therapy, mediated by lysine was instituted. The patient was ready for skin graft by around 12^{th} day post institution of lysine therapy (clinical end point). There was no other associated medication (antibiotic coverage) in the experimental category during the entire period of lysine therapy.

Cellulitic/Infected Wounds

Presented as an example here is a third degree burn (full thickness skin was lost) getting infected because of poor hygienic conditions, (so much prevalent in the developing countries). The infected wound was treated with conventional therapy of enzymatic debridement and dressings at regular intervals (daily) with surface antibiotics as well as other agents as outlined in the method section. Around 45 days' treatment gave rise to nearly no granulation tissue/angiogenic response. (High) lysine therapy alone brought about remarkable induction of granulation tissue/angiogenesis within around two weeks and clinical end point was reached (FIG. 6). The patient was ready for skin graft on day 13 post institution of therapy.

Burns

High lysine formulations, as was described in the earlier clinical conditions, were employed in the topical application treatment of burn cases of various degrees and extents (FIG. 7 and FIG. 8). Included in the comparative study is the conventional modes, of therapy (e.g. enzymatic debridement followed by dressings with salutyl, oxoferin, surface antibiotics, silversulfadiazine etc.). Remarkable degree of granulation tissue formation within around 72-96 hrs post institution of therapy with lysine was the constant feature.

Diabetic Ulcers and Wounds

High lysine formulations, as was described in the earlier clinical conditions were used in the topical application treatment of diabetic ulcers and wounds vis-à-vis the conventionally treated cases (FIG. 9). As in all other conditions, induction of angiogenesis leading to the healthy granulation tissue formation was remarkable in experimental cases compared to the controls. Histopathological examination showed development of hierarchical capillaries which got filled with blood cells around 10-12 days' time. Regeneration of healthy glandular structures was also obvious (FIG. 10).

Venous Ulcers and Ulcers and Wounds Due to Various Other Conditions

Wounds and ulcers of all anatomical locations and various durations were found to respond extremely favourably in terms of granulation tissue formation and angiogenic responses induced by lysine/derivative(s). Leprotic wound (˜3 yr old) showed very good granulation/angiogenesis and by about 12 weeks time (following topical treatment) showed ˜60% reduction in size. These wounds do not respond much to conventional therapy.

### Example 3

### Local Effect of Lysine Upon Angiogenesis In-Vivo

Topical applications of (high) lysine to chronic human wounds, as described in the Material and Method section, was effetive in inducing profuse angiogenic response as was made out from the histo-pathological examination of biopsy materials from lysine treated as well as controls (conventionally treated) (FIG. 11). Mature angiogenic buds appeared around 5^{th} to 6^{th} day (2^{nd} to 3^{rd} dressings) of institution of lysine therapy. Conventional modes of therapy induces angiogenesis and granulation tissue formation by 2-3weeks depending on the clinical status of the wound (Ref. Robbin's Basic Pathology, 7^{th} edition, June-2002, W. B Saunders, by Vinay Kumar, Ramji S Cotran and Stanley Robbins).

### Example 4

### Oligo-Lysine and D-Lysine Induced Cell Growth and Angiogenesis In-Vitro and In-Vivo

Lysine induced repair process is entirely dependent on the ability of the amino acid/derivative(s) to induce rapid and controlled cellular expansion, both in-vitro and in-vivo. The phenomenon is probably based on the ability of the molecule(s) to act as the cell surface concentrator(s) of circulating growth factor(s). The rapid angiogenic property of the molecule(s) is also entirely dependent on the same phenomenon, where angiogenic factors are concentrated, locally, on the endothelial cell surface receptors, mediated by the molecule(s) working, possibly ,as molecular bridges. Oligo-lysine and d-lysine are equally effective (as l-lysine) in inducing rapid cellular growth in-vitro (FIG. 12) and angiogenesis in-vivo (FIG. 13).

### Example 5

### Induction of Angiogenesis in Coronary Ischaemia Model

Induction of angiogenesis in the ischaemic myocardium was obvious, first, from physical agility and activity levels of the animals ˜120 hrs post surgery; secondly echocardiography done 96 hrs post surgery showed definite non-asynchronous movement of the myocardium in the ischaemic zones in experimental animals. This was in sharp contrast to the control animal where extent of ischaemic zone remained much wider around that time, compared to the experimental animals, coupled with much obvious asynchronous movement of the ischaemic myocardium. Histopathology showed advanced degree of matured angiogenic response in the experimental animals than in the control by around two-weeks when the experiment was terminated.

### Example 6

Few representative formulations can be as follows:

### Example

For application directly or by a mechanical spray.

| | |
|---|---|
| Lysine | 10 g |
| Methyl Paraben | 70 mg |
| Propyl Paraben | 30 mg |
| Purified Water | 89 9 g |
| | 100 0 g |

Solution for direct application or in the form of impregnated dressing:

| | |
|---|---|
| Lysine | 10.0 g |
| Methyl Paraben | 0.2 g |
| Propylene Glycol | 9.8 g |
| Purified water | 80.0 g |
| | 100.0 g |

Viscous solution for direct application (e.g. in the form of a lotion):

| | |
|---|---|
| Lysine | 10.0 g |
| Methyl Paraben | 0.1 g |
| Propylene Glycol | 8.0 g |
| Methyl Cellulose (1500) | 4.0 g |
| Purified Water | 77.9 g |
| | 100.0 g |

Application as water-miscible gel or hydrophilic gel

| | |
|---|---|
| Lysine | 10.0 g |
| Carboxymethyl Cellulose | 4.0 g |
| (sodium) | |
| Methyl Paraben | 0.1 g |
| Propylene Glycol | 22.0 g |
| Purified Water | 63.9 g |
| | 100.0 g |

Water miscible water-free gel form of application:

| | |
|---|---|
| Lysine | 10.0 g |
| Macrogol (3000) | 40.0 g |
| Macrogol (400) | 50.0 g |
| | 100.0 g |

Hydrophobic ointment form of application:

| | |
|---|---|
| Lysine | 10.0 g |
| Polyethylene | 8.5 g |
| Liquid Paraffin | 81.5 g |
| | 100.0 g |

Water absorbing ointment form of application

| | |
|---|---|
| Lysine | 10.0 g |
| Cetostearyl alcohol | 27.5 g |
| Polysorbate 80 | 2.5 g |
| Petroleum jelly | 50.0 g |
| Liquid paraffin | 10.0 g |
| | 100 0 g |

Cream form for application:

| | | |
|---|---|---|
| (A) | Lysine | 10.0 g |
| (B) | Polysorbate 80 | 0.45 g |
| | Cetyl alcohol | 4.5 g |
| | Liquid paraffin | 4.5 g |
| | Glycerol monostearate | 5.4 g |
| | 40-50 | |
| (C) | Glycerol 85% | 4.0 g |
| | Sorbitol | 6.0 g |
| | Methyl Paraben | 0.1 g |
| | Purified water | 65 15 g |

(B) is mixed and melted together at around 65-70° C. (C) is heated to boiling point and cooled to 70° C. approximately and mixed with (B). 10 grams of Lysine (A) of a particular particle size (<300 μm), put in 90.0 gram of the mixture of (B) and (C) Enhancement of active ingradient concentration to more than 15% might result in appearance of particulate matters in the bulk. This can be overcome by using hydrophilic polymer e.g. PVP.

Hydrophobic gel form (water immiscible)

| | |
|---|---|
| Lysine | 10.0 g |
| White soft paraffin | 75.0 g |
| Liquid paraffin | 15.0 g |
| | 100.0 g |

Dry powder form for direct application:

| | |
|---|---|
| Lysine | 10.0 g |
| Boric acid | 90.0 g |
| | 100.0 g |

In the form of tablets for rapid dissolution in intestinal tract for wound repair and/or ischaemic tissue angiogenesis

| | |
|---|---|
| Lysine | 500 mg |
| Cellulose (microcrystalline) | 100 mg |
| Povidone | 18 mg |
| Magnesium Stearate | 5 mg |
| Talc | 27 mg |
| | 550 mg |

Tablet formulation for oral application for wound healing and/or angiogenesis;

| | |
|---|---|
| Lysine | 500 mg |
| Calcium monohydrogen | |
| Phosphate | 82 mg |
| Croscaramellose Sodium | 25 mg |
| Povidone | 18 mg |
| Magnesium Stearate | 5 mg |
| Talc | 27 mg |
| | 657 mg |

Injectable form for ischaemic tissue and angiogenesis and/or wound repair:

| | |
|---|---|
| Lysine (and/or analogues/derivatives/oligomer(s) and/or | 100 mg/ml |
| salts thereof, as in example above) (in purified deionized | |
| water of injection grade) | |

[The total concentration of the active ingradient(s) in example, should be between 5-500 mg/ml, preferably between 20-200 mg/ml, more preferably between 50-100 mg/ml and most preferably about 100 mg/ml]

### Example 7:

To prove the long time cosmetic effect of Lysine induced quality of healing SEM of 4-month-old experimental wound with L-lysine HCl preparation applied topically was compared with 4-month-old control. Extensive deformation of the control wound compared to the experimental healing was observed. SEM analysis of tissue just below the surface wound (4 month old) in experimental wound and tissue below the control (deformed) wound showed substantial non-specific fibrosis in tissues below the control wounds, resulting in scar retraction whereas experimental wound showed a high degree of absence of non-specific fibrosis. This non-specific fibrosis in control wound causes downward pulling of surface tissue resulting in contracture and deformation. The wound-healing agent in its topically applicable forms is therefore of value in the prevention of scarring in primary and chronic wounds. (Illustrated in FIGS. 15(a-h). Most preferred optimum amounts incorporated in the experimental wounds, at the time of surgery (4 months back) ranged between 0.25 to 100 mg/sq. in of the wound.

[image]

[image]

Degree of non-specific fibrosis in the wound area was much more in control wounds compared to lysine mediated healing. Deformation and scarring of healed primary wounds in absence of lysine was substantial compared to lysine augmented healing. This scarring and deformation in the control wound was the result of non-specific (non-orderly); fibrous tissue net formation below the wound area. This was totally absent in lysine treated wounds.

### Example 8:

As glycine is present at every third position in the primary sequence of Collagen, ¹⁴C-Glycine incorporation in the wound tissue was supposed to give an idea of differential collagen synthesis in experimental and control wounds. Free skin glycine incorporation from control and experimental animals indicated a basal incorporation and collagen synthesis while in control and experimental wounds, glycine incorporation showed enhancement compared to non wounded skins respectively. Comparatively low incorporation in experimental wound in presence of L-lysine HCl 100% (w/w) applied topically (in the most preferred optimum amount of 0.25 to 100 mg/sq. in) was noted (illustrated in FIG. 16).

[image]

Result:

¹⁴C-Glycine incorporation was a distinct and definite way of showing the degree of collagen formation in the wound areas versus non wound areas in the two groups. Clearly, ¹⁴C-Glycine incorporation pattern (data) corroborated well with lysine induced less non-specific fibrosis resulting in less scarring.

### Example 9:

Non-specific protein synthesis pattern following topical application of 100% (w/w) powder formulation to acute clean-cut surgical wound was exhibited by ³⁵S-Methionine incorporation profile 72 hrs post surgery in both experimental and control wounds (no application). Significant difference in the ³⁵S-Methionine incorporation in the experimental wound tissue was noted indicating a probable surge in overall enhancement in protein synthesis, required for rapid cellular division in situ.

[image]

Results:

Significantly more ³⁵S-Methionine incorporation in amino acid-treated (0.25 to 100 mg/sq.in) wound area indicated enhanced synthesis of protein overall. This is a basic requirement of rapid cell division and expansion in the wound bed which is one of the bench marks of the present invention.

While the invention has been described in detail and with reference to the specific embodiments thereof, it will be apparent to one skilled in the art that various changes and modifications can be made therein without deviating or departing from the spirit and scope of the invention, essence of which lies in the fact that significant improvements of therapy are brought about while lowering the cost of therapy very very significantly. Thus the disclosure contained herein includes within its ambit the obvious equivalents and substitutes as well.

Having described the invention in detail with particular reference to the illustrative examples given above, it will now be more specifically defined by means of claims appended hereafter.

What is claimed is:-

1. A method of treating subjects having an ischaemic condition that may be treated by stimulating angiogenesis, the method comprising administering to a mammal (and human subjects) a cationic amino acid (either l- or d-form) and/or its oligomer (M.W. between 500 and 2500 ), either in native/ordinary form(s) or in activated form(s), in an amount, that stimulates or augments the formation of angiogenic buds and/or infiltration of optimal amount of inflammatory cells in or around the zone of application of the said cationic amino acid(s) and/or derivative(s), in ischaemic tissues.

2. The method of claim 1. wherein the cationic amino acid is l-lysine and/or d-lysine and/or oligo-lysine, arginine or mixture(s) thereof.

3. The method of claim 1, wherein said administration is topical, intravenous, intra-arterial, intra-pericardial or oral.

4. The method of claim 1, wherein said administering is to a local site.

5. The method of claim 1, wherein said administration is systemic and/or transdermal and/or intramuscular.

6. The method of claim 1, wherein said administration is for stimulation/induction of angiogenesis after surgery, elective or otherwise, resulting in scarless healing.

7. The method of claim 3, wherein said administration is topical.

8. The method of claim 6, wherein said administration is done at a site of an anastomosis, suture line or acute surgical wound.

9. The method of claim 1, the method further comprising administering the said agent(s) that enhance(s) accumulation of circulating angiogenic factor(s) on to the endothelial cell surface(s).

10. The method of claim 1, the method further comprising administering the said agent(s) that stimulate(s) local autocrine effect(s) of liberated angiogenic factor(s) in ischaemic tissue(s).

11. The method of claim 1, the method further comprising administering the said agent(s) that stimulate(s)/augment(s) and/or induce(s) general and overall protein synthesis in the wound bed.

12. The method of claim 1, the method further comprising administering the said agent(s) that enhance(s) in-situ cellular expansion/division in the wound bed (repair by in-situ regeneration).

13. The method of claim 1, the method further comprising administering the said agent(s) that optimizes formation of matrix components like collagen, elastin etc. in the wound bed resulting in optimal laying down of the matrix material(s) thereby preventing scarring and deformation.

14. The method of claim 1, wherein said administration is effective to stimulate/induce scarless healing in acute as well as chronic wounds by inducing/stimulating/augmenting the process of epithelialization, particularly in burns of various degrees and extents.

15. The method of claim 1, the method further comprising administering the said agent(s) that stimulate(s)/augment(s) the infiltration of blood cells in the newly formed capillaries.

16. The method of claim 1, wherein said administration is effective to stimulate angiogenesis in or around chronic wound(s) (e.g. diabetic wounds, pressure sores, leprotic wounds, venous ulcers, burns, geriatric wounds etc.)

17. The method of claim 1, wherein said administration is effective to stimulate angiogenesis in or around an ulcer/wound, skin graft or transplanted tissue resulting in a scarless healing.

18. The method of claim 1, wherein said administration is effective in regenerating healthy skin in human subjects without requirement of any graft procedure.

19. The method of claim 1, wherein said administration is effective in repair-by-in-situ-regeneration.

20. The method of claim 1, wherein said administration(s) through appropriate route(s) and combination(s), is effective to induce/stimulate and/or augment scarless healing in internal organ(s) e.g. neural tissue, cardiac tissue etc.

21. The method of claim 1, wherein said administration is effective to stimulate angiogenesis and formation of granulation tissue in chronic ischaemic wound(s).

22. The method of claim 1, wherein said administration is effective to stimulate angiogenesis and reperfusion in ischaemic myocardium, due to any reason(s), when administered through one or more of the following route(s) of administration—intracardiac (e.g. through angiography catheter mixed with or without the dye and/or through intramuscular injection route or a combination of the two approaches), parenteral (e.g. i.v.) or oral.

23. The method of claim 1, wherein said administration of effective dose of the said agent(s) is done without any other antibiotic or any other compound(s) for prevention of wound infection.

24. The method of claim 1, wherein the said administration(s) of the said agent(s) either alone or in various combination(s) through one or more route(s) of application(s), bring(s) about enhanced degree of angiogenesis and reperfusion in ischaemic cerebral/neural tissue(s), resulting in faster recovery in paralytic/spastic neural disorders (e.g. cerebral stroke due to any of the causative factors etc.).

25. The method of claim 1, wherein administration of the said agent(s), alone or in various combination(s), either through single or multiple route(s) of administration(s), results in enhanced reperfusion of ischaemic tissue (ischaemic muscle tissue) in conditions of peripheral limb ischaemia, due to various reasons.

26. The method of claim 1, wherein the said agent(s) is administered in a composition comprising one or more of the said agent(s) and a pharmacologically acceptable carrier with or without other additive(s) e.g. adjuvant(s)/stabilizer(s)/potentiator(s) etc. either for the purpose of ischaemic tissue reperfusion (cardiac/cerebral/limb etc.) and/or scarless healing of acute and chronic wounds, burns (even up to 3^{rd} degree) etc.

27. The method of claim 1, wherein the cationic amino acid/derivative is selected from lysine, arginine, ornithine, cadaverine etc. or pharmaceutically acceptable salts thereof.
